(19) United States
(12) Patent Application Publication
(10) Pub. No.: US 2021/0340557 A1
(43) Pub. Date: Nov. 4, 2021

(54) BEET NECROTIC YELLOW VEIN VIRUS (BNYVV)-RESISTANCE MODIFYING GENE

(71) Applicant: KWS SAAT SE & Co. KGaA, Einbeck (DE)

(72) Inventors: Ann-Katrin MÜNNEKHOFF, Einbeck (DE); Britta SCHULZ, Einbeck (DE); Daniel Fabian STIRNWEIS, Gottingen (DE); Ottó TÖRJÉK, Einbeck (DE); Dietrich BORCHARDT, Einbeck (DE)

(73) Assignee: KWS SAAT SE & Co. KGaA, Einbeck (DE)

(21) Appl. No.: 17/274,692

(22) PCT Filed: Sep. 11, 2019
(86) PCT No.: PCT/EP2019/074285

§ 371 Date: Mar. 9, 2021

(30) Foreign Application Priority Data
Sep. 11, 2018 (EP) 18193892.9

(51) Int. Cl. C12N 15/82; C07K 14/415; C12Q 1/6895

(52) U.S. Cl. CPC C12N 15/8283; C07K 14/415; C12Q 2600/156; C12Q 2600/13; C12Q 1/6895

(57) ABSTRACT

A method for establishing, restoring or increasing the Rz2 gene mediated resistance to the Beet necrotic yellow vein virus (BNYVV) in the target plant is enabled via the identification and provision of a gene which modulates said resistance according to the invention. In particular, the BNYVV resistance mediated by the Rz2 resistance gene depends on the presence of the gene of the present invention. The gene being involved in conferring said resistance, and embodiments of the present invention that are described in the preceding, offer additional applications, e.g., the use of the resistance modifying gene allele in cis-genetic or trans-genetic approaches, with the goal of developing new resistant cultivars.

### FIELD OF THE INVENTION

The present invention relates to a nucleic acid molecule which encodes a polypeptide, which is involved in Rz2 resistance gene mediated resistance against Beet necrotic yellow vein virus (BNYVV) in a plant in which the polypeptide is expressed as well as to a nucleic acid molecule which encodes a non-functional allele of the polypeptide, which interferes with said resistance against BNYVV in a plant in which the polypeptide is expressed. The present invention further relates to the corresponding polypeptides encoded by the nucleic acid molecules according to the invention. Furthermore, the invention relates to a plant, plant cell, plant organ, plant tissue, plant part, or a seed or descendant of a plant, which comprises any one of the nucleic acid molecules or portions thereof as an endogenous gene or as a transgene. Furthermore, the present invention also encompasses methods for conferring, restoring or increasing the resistance to BNYVV in a plant of the species Beta vulgaris comprising the Rz2 resistance gene, as well as methods for producing or identifying and possibly selecting a BNYVV-resistant plant. The present invention also encompasses methods for cultivating plants of the species Beta vulgaris counteracting an infestation of the cultivated plants with BNYVV as well as to oligonucleotide probes and primers for specific hybridization with any one of the nucleic acid molecules according to the invention.

### BACKGROUND OF THE INVENTION

Rhizomania is the most economically important sugar beet disease worldwide, which can cause yield losses of 50% or more. The disease, also called “root madness”, is caused by the Beet necrotic yellow vein virus (BNYVV) and transmitted by the soil-borne fungus Polymyxa betae. BNYVV infection manifests itself in an increased proliferation of the thin roots and lateral roots and in the formation of a strongly reduced root body with reduced sugar content. Infected plants show a reduced water absorption and are therefore more sensitive to drought stress. When the infection spreads to the entire plant, the leaf veins become yellow, with necrotic lesions and yellow spots on the leaves. Since curative control of the disease is not possible, as with other viral diseases, damage can only be prevented by cultivating resistant varieties. Three major genes are currently under investigation against rhizomania: Rz1 (also known as “Holly”), Rz2 and Rz3.

The Rz2 resistance gene, which was identified and described in international patent application WO 2014/202044 A1 originates from the Beta maritima accession WB42 and is known to provide an elevated resistance to rhizomania in comparison to Rz1 when expressed in several varieties of sugar beet. Furthermore, it is known that Rz3 from Beta maritima accession WB41 has been identified, which is most probably the same or a very similar allelic variant of Rz2.

However, in the course of development of new sugar beet varieties, it has been observed that certain genetic backgrounds lead to a weakening or loss of resistance, although the resistant allele of Rz2 or Rz3 is present. So far, nothing is known about the molecular genetic background leading to the observed effect. However, in order to make the resistance effect mediated by Rz2 or Rz3, respectively, usable in all genetic backgrounds, there is a high demand among plant breeders of the genus Beta to prevent the observed reduction or loss of resistance.

In particular, the aim is to identify the mechanism behind the observed reduction or loss of resistance and to use it in order to establish sugar beet lines which show a stable resistance to rhizomania based on Rz2 and Rz3, respectively. According to the invention, this aim is achieved via the embodiments characterized in the claims and in the specification.

### SUMMARY OF THE INVENTION

The present invention relates to a nucleic acid molecule which encodes a polypeptide, which is involved in resistance against Beet necrotic yellow vein virus (BNYVV) mediated by the Rz2 resistance gene in a plant as well as to a nucleic acid molecule which encodes a non-functional allele of the polypeptide, which interferes with said resistance against BNYVV in which the polypeptide is expressed. The polypeptide which is encoded by any one of the nucleic acid molecules is thereby produced in the plant.

Furthermore, the invention relates to a plant, plant cell, plant organ, plant tissue, plant part, or a seed or descendant of a plant, which endogenously or transgenically comprises any one of the nucleic acid molecules or portions thereof. According to a specific optional embodiment, those plants and their components that have been obtained via an essentially biological process are exempted.

Methods for conferring, restoring or increasing the resistance to BNYVV in a plant of the species Beta vulgaris comprising the Rz2 resistance gene, as well as methods for producing or identifying and possibly selecting a BNYVV-resistant plant, are likewise encompassed by the present invention. The present invention also encompasses methods for cultivating plants of the species Beta vulgaris counteracting an infestation of the cultivated plants with BNYVV as well as oligonucleotide probes and primers for specific hybridization with any one of the nucleic acid molecules according to the invention.

The present invention therefore relates to the embodiments that are listed in the following points [1] through [29] and illustrated in the Examples and Figures.
[1] A nucleic acid molecule encoding a polypeptide which is involved in resistance against Beet necrotic yellow vein virus (BNYVV) mediated by the Rz2 resistance gene in a plant in which the polypeptide is expressed, wherein the nucleic acid molecule comprises a nucleotide sequence which is selected from the group consisting of:
   (a) a nucleotide sequence that encodes a polypeptide having the amino acid sequence according to SEQ ID No. 45;
   (b) a nucleotide sequence that comprises the coding DNA sequence according to SEQ ID No. 44
   (c) a nucleotide sequence that hybridizes with a complementary sequence of the nucleotide sequence according to (a) or (b) under stringent conditions;
   (d) a nucleotide sequence that encodes a polypeptide which, via substitution, deletion, and/or addition of one or more amino acids of the amino acid sequence, differs from a polypeptide that is encoded by the nucleotide sequence according to (a) or (b);
   (e) a nucleotide sequence that encodes a polypeptide which has an amino acid sequence that is at least 70% identical to the amino acid sequence according to SEQ ID No. 45; and
   (f) a nucleotide sequence that comprises the coding DNA sequence which is at least 70% identical to the DNA sequence according to SEQ ID No. 44,
   wherein the amino acid sequence of the encoded polypeptide has a proline (P) at position 25 with reference to SEQ ID No. 45, a proline (P) at position 72 with reference to SEQ ID No. 45 and/or a valine (V) at position 186 with reference to SEQ ID No. 45, or
   wherein the nucleic acid of the coding DNA sequence has a cytosine (c) at position 73 with reference to SEQ ID No. 44, a cytosine (c) at position 214 with reference to SEQ ID No. 44, and/or a guanine (g) at position 556 with reference to SEQ ID No. 44.
[2] The nucleic acid molecule according to [1], wherein the polypeptide has the amino acid sequence of SEQ ID No. 8, 12, 16, or 20, or the nucleotide sequence that encodes said polypeptide is SEQ ID No. 5, 6, 7, 9, 10, 11, 13, 14, 15, 17, 18 or 19, or the coding DNA sequence is a nucleotide sequence according to SEQ ID No. 7, 11, 15, or 19.
[3] A nucleic acid molecule which specifically hybridizes to the nucleic acid molecule according to [1] or [2] and encodes a non-functional allele of the polypeptide, which interferes with resistance against BNYVV mediated by the Rz2 resistance gene in a plant in which the polypeptide is expressed, wherein the nucleic acid molecule preferably comprises a nucleotide sequence which is selected from
   (a) a nucleotide sequence that encodes a polypeptide having an amino acid sequence according to SEQ ID No. 4 or a polypeptide having an amino acid sequence that is at least 70% identical to SEQ ID No. 4, wherein the polypeptide comprises an amino acid sequence having at least one amino acid substitution due to one or more mutations in the nucleotide sequence, preferably wherein proline (P) at position 25 with reference to SEQ ID No. 45, proline (P) at position 72 with reference to SEQ ID No. 45, and/or valine (V) at position 186 with reference to SEQ ID No. 45 is substituted with another amino acid; and/or
   (b) a nucleotide sequence that comprises the coding DNA sequence according to SEQ ID No. 3, that comprises a coding DNA sequence which is at least 70% identical to the DNA sequence according to SEQ ID No. 3, or that hybridizes with a complementary sequence of SEQ ID No. 3 under stringent conditions, wherein the nucleotide sequence comprises at least one nucleic acid substitution due to at least one mutation leading to an amino acid substitution, preferably wherein one or more nucleotides are substituted at positions 73-75 with reference to SEQ ID No. 44, at positions 214-216 with reference to SEQ ID No. 44 and/or at positions 556-558 with reference to SEQ ID No. 44.
[4] The nucleic acid molecule according to [3], characterized in that the encoded polypeptide comprises an amino acid sequence having
   (i) alanine (A) instead of proline (P) at position 25 with reference to SEQ ID No. 45,
   (ii) threonine (T) instead of proline (P) at position 72 with reference to SEQ ID No. 45, and/or
   (iii) isoleucine (I) instead of valine (V) at position 186 with reference to SEQ ID No. 45.
[5] The nucleic acid molecule according to [3] or [4], characterized in that the nucleic acid molecule comprises a DNA sequence having
   (i) guanine (g) instead of cytosine (c) at position 73 with reference to SEQ ID No. 44,
   (ii) adenine (a) instead of cytosine (c) at position 214 with reference to SEQ ID No. 44, and/or
   (iii) adenine (a) instead of guanine (g) at position 556 with reference to SEQ ID No. 44.
[6] The nucleic acid molecule according to any one of [3] to [5], characterized in that the nucleic acid molecule comprises a coding DNA sequence having in addition
   (i) adenine (a) instead of guanine (g) at that position 72 with reference to SEQ ID No. 44, and/or
   (ii) adenine (a) instead of guanine (g) at position 111 with reference to SEQ ID No. 44.
[7] A polypeptide which is encoded by the nucleic acid molecule according to [1] or [2] or any one of [3] to [6].
[8] A vector or expression cassette which comprises the nucleic acid molecule according to [1] or [2] or any one of [3] to [6], preferably wherein the nucleic acid molecule according to [1] or [2] or any one of [3] to [6] is operably linked to a heterologous regulatory element, preferably wherein the regulatory element is a promoter.
[9] A cell which comprises the nucleic acid molecule according to [1] or [2] or any one of [3] to [6], a polypeptide according to [7], or a vector or expression cassette according to [8].
[10] A plant or a portion thereof comprising the nucleic acid molecule according to [1] or [2] endogenously or transgenically or the vector or the expression cassette which comprises the nucleic acid molecule according to [1] or [2] as defined in [8] as transgene.
[11] A plant or a portion thereof according to [10], characterized by comprising additionally the Rz2 resistance gene mediating BNYVV resistance endogenously or transgenically.
[12] Seed or a descendant of the plant according to [10] or [11], wherein the seed or the descendant endogenously or transgenically comprises the nucleic acid molecule according to [1] or [2], or the vector or the expression cassette according to [8].
[13] Seed according to [12], which has been technically treated, whereby the technical treatment is selected from the group consisting of:

(a) Polishing,

(b) Dressing, preferably pelleting,

(c) Incrustation, and

(d) Colouring.
[14] Oligonucleotide or a pair of oligonucleotides of at least 15, 16, 17, 18, 19, or 20, preferably at least 21, 22, 23, 24, or 25, particularly preferably at least 30, 35, 40, 45, or 50, and particularly preferably at least 100, 200, 300, or 500 nucleotides in length, wherein the oligonucleotide or the pair of oligonucleotides
   (i) specifically hybridizes to a nucleotide sequence as defined in any one of [3] to [6], but not to a nucleotide sequence as defined in [1] or [2];
   (ii) specifically hybridizes to a nucleotide sequence as defined in [1] or [2], but not to the nucleotide sequence as defined in any one of [3] to [6];
   (iii) specifically hybridizes to a region in the Beta vulgaris genome that, in Beta vulgaris, cosegregates with the nucleic acid molecule according to any one of [3] to [6] and/or with the nucleic acid molecule according to any one of [3] to [6] together with the Rz2 gene; and/or
   (iv) specifically hybridizes to a region in the Beta vulgaris genome that, in Beta vulgaris, cosegregates with the nucleic acid molecule according to [1] or [2] and/or with the nucleic acid molecule according to [1] or [2] together with the Rz2 gene.
[15] The oligonucleotide or pair of oligonucleotides according to [14], wherein the oligonucleotide or pair of oligonucleotides is selected from the group consisting of SEQ ID NOs: 24 to 40.
[16] A method for conferring, restoring or increasing the resistance to BNYVV in a plant of the species Beta vulgaris comprising the Rz2 resistance gene mediating BNYVV resistance endogenously or transgenically, including the following steps:
   (i) integration or introgression of the nucleic acid molecule according to [1] or [2] by means of homology-directed repair or homologous recombination—preferably, promoted by site-directed nuclease—into the genome of at least one cell of a plant of the species Beta vulgaris, and optional regeneration of a plant from the plant cell; or
   (ii) increasing the expression of the nucleic acid molecule according to [1] or [2] in the plant—preferably, via modification of the native promoter or via fusion of the nucleic acid molecule with a heterologous promoter that exhibits a higher activity in comparison to the native promoter—in particular, after BNYVV infection; or
   (iii) increase in the activity and/or stability of the functional allele of the polypeptide which is encoded by the nucleic acid molecule according to [1] or [2] as defined in [7] via modification of the nucleotide sequence according to [1] or [2];
   (iv) transformation of a plant cell with the nucleic acid molecule according to [1] or [2], or the vector or the expression cassette which comprises the nucleic acid molecule according to [1] or [2] as defined in [8,] and optionally regeneration of a transgenic plant from the transformed plant cell; or
   (v) mutagenesis of a plant or plant cells comprising the nucleic acid molecule according to any one of [3] to [6], and screening/selecting for plants or plant cells, respectively, in which a functional allele of the polypeptide encoded by the nucleic acid molecule according to [1] or [2] has been restored and/or the expression of the non-functional polypeptide has been abolished.
[17] A method for producing a BNYVV-resistant plant comprising the nucleic acid molecule according to [1] or [2] as defined in [10], including the following steps:
   (I) provision of a plant of the genus Beta or a plant cell of the genus Beta comprising the Rz2 resistance gene mediating BNYVV resistance endogenously or transgenically; and
   (IIa) transformation of the plant cell of (I) with the nucleic acid molecule according to [1] or [2], or the vector or the expression cassette which comprises the nucleic acid molecule according to [1] or [2] as defined in [8]; and
   (IIb) regeneration of a transgenic plant from the transformed plant cell; or
   (IIIa) introduction of a site-directed nuclease or nickase and optionally a repair matrix, or a site-directed base editor into the plant cell, wherein the site-directed nuclease is able to generate at least one single-strand break of the DNA or at least one double-strand break of the DNA at a predetermined location in the genome of the cell and the repair matrix comprises the nucleic acid molecule according to [1] or [2], or wherein the site-directed base editor is able to generate at least one single-strand break of the DNA at a predetermined location in the genome of the cell and to convert at least one nucleobase; and
   (IIIb) optionally, cultivation of the cell from (IIIa) under conditions that allow modification of the genome at the predetermined location, selected from
   a) a replacement of at least one nucleotide;
   b) a deletion of at least one nucleotide;
   c) an insertion of at least one nucleotide;
   d) any combination of a)-c),
   optionally by homology-directed repair or homologous recombination,
   wherein the nucleic acid molecule is integrated into the genome of the plant; and
   (IIIc) regeneration of a plant from the cell modified in (IIIb); or
   (IVa) crossing of the plant of (I) with the plant according to any one of [10] to [12]; and
   (IVb) identifying and selecting a plant comprising the Rz2 resistance gene mediating BNYVV endogenously or transgenically and the nucleic acid molecule according to [1] or [2] endogenously or transgenically, wherein the Rz2 resistance gene and/or the nucleic acid molecule according to [1] or [2] is present homozygously in the genome of the plant.
[18] The method according to [17], characterized in that the predetermined location is in the nucleic acid molecule according to any one of [3] to [6] or a position which is at most 10.000 base pairs upstream or downstream away from the nucleic acid molecule according to any one of [3] to [6].
[19] A method for identifying a plant of the species Beta vulgaris, a portion thereof or a seed thereof comprising the nucleic acid molecule according to [1] or [2], wherein the plant is capable of exhibiting resistance against BNYVV mediated by Rz2, if present in the genome of the plant, comprising:
   (i) detection of the presence and/or expression of the nucleic acid molecule according to [1] or [2], or the presence of the polypeptide which is encoded by the nucleic acid molecule according to [1] or [2] as defined in [7] in the plant, the portion thereof or the seed thereof; and/or
   (ii) detection of at least one marker locus in the nucleotide sequence of the nucleic acid molecule according to [1] or [2] or in one or more cosegregating regions thereof; and
   (iii) identification and optionally selection of the plant of the species Beta vulgaris, the portion thereof or the seed thereof comprising the nucleic acid molecule according to [1] or [2], which is capable of exhibiting resistance against BNYVV mediated by Rz2, if present in the genome of the plant, based on the detection of (i) and/or (ii).
[20] A method for identifying a plant of the species Beta vulgaris, a portion thereof or a seed thereof comprising the nucleic acid molecule according to any one of [3] to [6], comprising:
   (i) detection of the presence and/or expression of the nucleic acid molecule according to any one of [3] to [6], or the presence of the polypeptide which is encoded by the nucleic acid molecule according to any one of [3] to [6] as defined in [7] in the plant, the portion thereof or the seed thereof; and/or
   (ii) detection of at least one marker locus in the nucleotide sequence of the nucleic acid molecule according to any one of [3] to [6] or in one or more cosegregating regions thereof; and
   (iii) identification and optionally selection of the plant of the species Beta vulgaris, the portion thereof or the seed thereof comprising the nucleic acid molecule according to any one of [3] to [6], based on the detection of (i) and/or (ii).
[21] The method according to [19] or [20], wherein the detection of the at least one marker locus in the nucleotide sequence of the nucleic acid molecule according to [1] or [2] or in the nucleotide sequence of the nucleic acid molecule according to any one of [3] to [6] comprises the use of the oligonucleotide or the pair of oligonucleotides according to claim 14 or 15, and wherein the one or more cosegregating regions are genomic intervals in Beta vulgaris on chromosome 3 which comprise and are flanked by marker loci detectable by means of
   (i) the markers s3e5985s01 (SEQ ID No. 33) and s3p4348s01 (SEQ ID No. 27), and/or
   (ii) the markers s3p4351s01 (SEQ ID No. 29) and s3e2247xxx (SEQ ID No. 40), preferably by means of
   (i) the markers sxh1002s02 (SEQ ID No. 34) and s3p4348s01 (SEQ ID No. 27), and/or
   (ii) the markers s3p4351s01 (SEQ ID No. 29) and sxn1017s02 (SEQ ID No. 39), more preferably means of
   (i) the markers sxh2499s01 (SEQ ID No. 35) and s3p4348s01 (SEQ ID No. 27), and/or
   (ii) the markers sxi0698s01 (SEQ ID No. 36) and s3p4348s01 (SEQ ID No. 27), and/or
   (iii) the markers s3p4351s01 (SEQ ID No. 29) and sxe7357s01 (SEQ ID No. 38), and/or
   (iv) the markers s3p4351s01 (SEQ ID No. 29) and sxh8485s01 (SEQ ID No. 37), and/or
   (v) the markers s3p4351s01 (SEQ ID No. 29) and s3e5853s01 (SEQ ID No. 26), and/or
   (vi) the markers s3p4351s01 (SEQ ID No. 29) and s3e5800s01 (SEQ ID No. 32), and/or
   (vii) the markers s3p4351s01 (SEQ ID No. 29) and s3e4913xxx (SEQ ID No. 31), and/or
   (viii) the markers s3p4351s01 (SEQ ID No. 29) and s3e5782s01 (SEQ ID No. 30).
[22] The method according to [19] or [21] additionally comprising
   (A) detection of the presence and/or expression of the Rz2 gene or the presence of the polypeptide encoded by the Rz2 gene in the plant, the portion thereof or the seed thereof; and/or
   (B) detection of at least one marker locus in the Rz2 gene or in one or more regions cosegregating with the Rz2 gene, preferably cosegregating with a chromosomal interval comprising the nucleic acid molecule according to [1] or [2] and the Rz2 gene;
   wherein in (iii) of [19] the identified and optionally selected plant, the portion thereof or the seed thereof comprises the nucleic acid molecule according to [1] or [2], and the Rz2 gene, based on the detection of (i) and/or (ii) of [19] and detection of (B).
[23] The method according to [22], wherein the at least one marker locus in the nucleotide sequence of the Rz2 gene is detectable by means of the marker s3e5853s01 (SEQ ID No. 26) and wherein the one or more regions cosegregating with the Rz2 gene are genomic intervals in Beta vulgaris on chromosome 3 which comprise and are flanked by marker loci detectable by means of
   (i) the markers s3p4351s01 (SEQ ID No. 29) and s3e5853s01 (SEQ ID No. 26), and/or
   (ii) the markers s3e5853s01 (SEQ ID No. 26) and s3e2247xxx (SEQ ID No. 40), preferably by means of
   (i) the markers s3p4351s01 (SEQ ID No. 29) and s3e5853s01 (SEQ ID No. 26), and/or
   (ii) the markers s3e5853s01 (SEQ ID No. 26) and sxn1017s02 (SEQ ID No. 39), more preferably means of
   (i) the markers s3p4351s01 (SEQ ID No. 29) and s3e5853s01 (SEQ ID No. 26), and/or
   (ii) the markers s3e5853s01 (SEQ ID No. 33) and sxe7357s01 (SEQ ID No. 38), and/or
   (iii) the markers s3e5853s01 (SEQ ID No. 26) and sxh8485s01 (SEQ ID No. 37).
[24] The method according to [22], wherein the in one or more regions cosegregating with the chromosomal interval comprising the nucleic acid molecule according to [1] or [2] and the Rz2 gene are genomic intervals in Beta vulgaris on chromosome 3 which comprise and are flanked by marker loci detectable by means of
   (i) the markers s3e5985s01 (SEQ ID No. 33) and s3p4348s01 (SEQ ID No. 27), and/or
   (ii) the markers s3e5853s01 (SEQ ID No. 26) and s3e2247xxx (SEQ ID No. 40), preferably by means of
   (i) the markers sxh1002s02 (SEQ ID No. 34) and s3p4348s01 (SEQ ID No. 27), and/or
   (ii) the markers s3e5853s01 (SEQ ID No. 26) and sxn1017s02 (SEQ ID No. 39), more preferably means of
   (i) the markers sxh2499s01 (SEQ ID No. 35) and s3p4348s01 (SEQ ID No. 27), and/or
   (ii) the markers sxi0698s01 (SEQ ID No. 36) and s3p4348s01 (SEQ ID No. 27), and/or
   (iii) the markers s3e5853s01 (SEQ ID No. 26) and sxe7357s01 (SEQ ID No. 38), and/or
   (iv) the markers s3e5853s01 (SEQ ID No. 26) and sxh8485s01 (SEQ ID No. 37).
[25] Use of the oligonucleotide or pair of oligonucleotides according to [14] or [15] for identifying and/or selecting a plant of the species Beta vulgaris capable of exhibiting resistance to BNYVV mediated by Rz2, if present in the genome of the plant, or which capability of exhibiting resistance to BNYVV mediated by Rz2, if present in the genome of the plant, is impaired.
[26] A method for cultivation of plants of the species Beta vulgaris, including
   (i) planting seedlings of the plant according to [10] or [11] or sowing seeds according to [12] or [13], and
   (ii) grown the plants from the seedlings or seeds, and
   (iii) optionally, harvest root beets from the grown plants;
   wherein the method counteracts an infestation of the cultivated plants with BNYVV.
[27] Use of the nucleic acid molecule according to [1] or [2] or the nucleic acid molecule according to any one of [3] to [6], the polypeptide according to [7], the vector or expression cassette according to [8], and/or the cell according to [9] for the identification of nucleic acid molecules which are involved in Rz2 mediated resistance to BNYVV.

[28] Use of the nucleic acid molecule according to [1] or [2] or the nucleic acid molecule according to any one of [3] to [6], the polypeptide according to [7] and/or the vector or expression cassette according to [8] for the production of BNYVV-resistant plants, preferably of plants of the species Beta vulgaris, more preferably of plants of Beta vulgaris ssp. vulgaris var. vulgaris, Beta vulgaris ssp. vulgaris var. conditiva, oder Beta vulgaris ssp. vulgaris var. crassa/alba.
[29] The nucleic acid molecule according to [1] to [6], the plant or a portion thereof according to [11], the oligonucleotide or a pair of oligonucleotides according to [14] or [15], the methods according to any one of [16] to [19] and [22] to [24], and the use according to [25] and [27], wherein the Rz2 gene or the Rz2 resistance gene is a nucleic acid molecule comprises a nucleotide sequence selected from
   a) a nucleotide sequence that encodes a polypeptide with an amino acid sequence according to SEQ ID NO. 22 or SEQ ID NO. 23,
   b) a nucleotide sequence comprising the coding sequence of the DNA sequence according to SEQ ID NO. 21,
   c) a nucleotide sequence that encodes a polypeptide derived by substitution, deletion and/or addition of an amino acid of the amino acid sequence encoded by the nucleotide sequence according to a) or b), from a polypeptide encoded by the nucleotide sequence according to a) or b),
   d) a nucleotide sequence that encodes a polypeptide having an amino acid sequence, which is at least 80% identical to an amino acid sequence encoded by the nucleotide sequence according to a) or b), or
   e) a nucleotide sequence which encodes at least the amino acid positions 168-227 of SEQ ID NO. 22 and at least the amino acid positions 591-613 of SEQ ID NO. 22 and at least the amino acid positions 1013-1072 of SEQ ID NO. 22 or which encodes at least the amino acid positions 182-241 of SEQ ID NO. 23, at least the amino acid positions 605-627 of SEQ ID NO. 23 and at least the amino acid positions 1027-1086 of SEQ ID NO. 23.
[30] The method according to [19] wherein the detection according to step (i) and/or step (ii) is based on PCR, preferably kompetitive allele specific PCR (KASP) wherein optionally labeled primers are applied.
[31] The method according to [20] wherein the detection according to step (i) and/or step (ii) is based on PCR, preferably kompetitive allele specific PCR (KASP) wherein optionally labeled primers are applied.
[32] The method according to [26] wherein the planting according to (i) is done by a mechanical device which is preferably selected from the group consisting of: sower, planter and seeder.
[33] The method according to [26] or [32] wherein the harvest according to (iii) is done by a mechanical device which may be a harvester preferably a sugar beet harvester.
[34] The method according to [26], [32] or [33] wherein the leaves of the plant are removed mechanically during the harvest according to (iii) wherein the mechanical removal is preferably done by a harvester as defined in [33].
[35] The method according to [26], [32], [33] or [34] comprising an additional step (iv) extracting sugar, preferably sucrose, from the harvested root beets.
[36] The use according to [25] or the oligonucleotide or a pair of oligonucleotides according to [14] or [15] wherein the oligonucleotide or the pair of oligonucleotides is labeled preferably fluorescently labeled, more preferably fluorescently labeled by FAM or HEX.
[37] A cell extract of the cell according to [9], a plant or a portion thereof according to [10] or [11].
[38] The cell extract according to [37] comprising the nucleic acid molecule according to [1].
[39] The cell extract according to [37] or [38] wherein the cell extract comprises sucrose.
[40] The cell extract according to [37], [38] or [39] wherein the sucrose concentration is at least 10%, preferably at least 15% more preferably at least 20% and most preferably 10%-20% wherein the concentration is given as mass concentration [g/dl].
[41] The method according to [19] or [20] wherein the identified plant or its seed are used in a method according to [26], [32], [33] or [34].
[42] The polypeptide according to [7] wherein the protein comprises one or two or three or four or five amino acid substitutions within the sequence of SEQ ID No. 45.
[43] The polypeptide according to [7] wherein the polypeptide is an artificial compound which does not occur in nature.
[44] A method for the production of molecular markers for the identification of the nucleic acid molecule according to [1] comprising the following steps:
   a) Providing a sequence alignment between SEQ ID No. 54 and SEQ ID No. 55
   b) Identifying at least one polymorphism between SEQ ID No. 54 and SEQ ID No. 55
   c) Developing one more molecular markers which are suitable to detect the polymorphism identified under b)
[45] The method according to [44] wherein the at least one polymorphism is a single nucleotide polymorphism.
[46] Use of a molecular marker on a nucleotide sequence according to SEQ ID No. 54 for the identification of the nucleic acid molecule according to [1] or for the identification of a plant comprising the nucleic acid molecule according to [1].

First, some of the terms used in this application are explained in detail in the following:

In conjunction with the specification of a length of a nucleotide sequence, the term, “approximately,” means a deviation by +/−200 base pairs—preferably, by +/−100 base pairs, and, particularly preferably, by +/−50 base pairs.

A “plant of the genus Beta” belongs to the amaranth family (Amaranthaceae). Numbering among these plants are plants of the species Beta macrocarpa, Beta vulgaris, Beta lomatogona, Beta macrorhiza, Beta corolliflora, Beta trigyna, and Beta nana. A plant of the species Beta vulgaris is, in particular, a plant of the subspecies Beta vulgaris subsp. vulgaris. For example, numbering among these are Beta vulgaris subsp. vulgaris var. altissima (sugar beet in a narrower sense), Beta vulgaris ssp. vulgaris var. vulgaris (chard), Beta vulgaris ssp. vulgaris var. conditiva (beetroot/red beet), Beta vulgaris ssp. vulgaris var. crassa/alba (fodder beet).

A “functional fragment” of a nucleotide sequence means a segment of a nucleotide sequence which has a functionality identical or comparable to that of the complete nucleotide sequence from which the functional fragment originates. As such, the functional fragment may possess a nucleotide sequence which is identical or homologous to the total nucleotide sequence over a length of at least 50%, 55%, 60%, 65%, 70%, 75%, 80%, 85%, 90%, 92%, 94% 96%, 97%, 98%, or 99%. This also explicitly encompasses the range of 90-100%. Furthermore, a “functional fragment” of a nucleotide sequence may also mean a segment of a nucleotide sequence which modifies the functionality of the entire nucleotide sequence, e.g., in the course of post-transcriptional or transcriptional gene silencing. As such, the functional fragment of a nucleotide sequence may comprise at least 14, 15, 16, 17, 18, 19, 20, 21, 22, 23, 24, or 25—preferably, at least 30, 35, 40, 45, 50, 60, 70, 80, 90, 100, 120, or 140, and, particularly preferably, at least 160, 180, 200, 250, 300, 350, 400, 450, 500, 600, 700, 800, 900, or 1,000—successive nucleotides of the total nucleotide sequence. This also explicitly encompasses the range of 21 to 50 nucleotides.

A “functional part” of a protein means a segment of a protein, or a section of the amino acid sequence, that encodes the protein, wherein the segment may exert functionality identical or comparable to that of the entire protein in a plant cell. Over a length of at least 50%, 55%, 60%, 65%, 70%, 75%, 80%, 85%, 90%, 92%, 94% 96%, 97%, 98%, or 99%, a functional part of a protein has an amino acid sequence that is identical or, with consideration of conservative and semi-conservative amino acid exchanges, similar to the protein from which the functional part originates.

The term, “heterologous,” means that the introduced polynucleotide originates from a cell or an organism with a different genetic background, of the same species or a different species, or is homologous to the prokaryotic or eukaryotic host cell, but is then located in a different genetic environment and thus differs from a corresponding polynucleotide that is possibly naturally present. A heterologous polynucleotide may be present in addition to a corresponding endogenous gene.

In the sense of the invention, what is understood by a “homolog” is a protein of the same phylogenetic origin; what is understood by an “analog” is a protein which exerts the same function, but has a different phylogenetic origin; what is understood by an “ortholog” is a protein from a different species that exerts the same function; and what is understood by a “paralog” is a protein that has appeared within a species due to duplication, wherein this copy either retains the same protein function, alters its expression template, but not the function, changes its protein function, or divides up the original gene function between both copies.

What is to be understood by “hybridizing” or “hybridization” is a process in which a single-stranded nucleic acid molecule binds to a nucleic acid strand that is complementary to the greatest possible extent, i.e., forms base pairs with this. Standard methods for hybridization are described in, for example, Sambrook et al., Molecular Cloning: A Laboratory Manual, 3rd ed., Cold Spring Harbor Laboratory Press, Cold Spring Harbor, N.Y., 2001. What is preferably understood by this is that at least 60%—more preferably, at least 65%, 70%, 75%, 80%, or 85%, and, particularly preferably, 90%, 91%, 92%, 93%, 94%, 95%, 96%, 97%, 98%, or 99%—of the bases of the nucleic acid molecule form a base pairing with the nucleic acid strand that is complementary to the greatest possible extent. The possibility of such an annealing depends upon the stringency of the hybridization conditions. The term, “stringency,” relates to the hybridization conditions. High stringency is present when a base pairing is made more difficult; low stringency is present if a base pairing is made easier. For example, the stringency of the hybridization conditions depends upon the salt concentration or ionic strength and the temperature. In general, the stringency may be increased by increasing the temperature and/or decreasing the salt content. What are to be understood by “stringent hybridization conditions” are those conditions given which a hybridization predominantly occurs only between homologous nucleic acid molecules. The term, “hybridization conditions,” thereby relates not only to the conditions prevailing in the actual addition of the nucleic acids, but also to the conditions prevailing in the following washing steps. For example, stringent hybridization conditions are conditions under which, predominantly, only those nucleic acid molecules hybridize that have at least 70%—preferably, at least 75%, at least 80%, at least 85%, at least 90%, or at least 95%—sequence identity. Stringent hybridization conditions are, for example: hybridization in 4×SSC at 65° C., and subsequent repeated washing in 0.1×SSC at 65° C. for approximately 1 hour in total. A hybridization preferably occurs under stringent conditions.

In relation to a nucleic acid in the form of a double-stranded DNA, “complementary” nucleotide sequence means that the second DNA strand complementary to the first DNA strand has the nucleotides that correspond to the bases of the first strand, in accordance with the base pairing rules. A complementary sequence is, preferably, entirely complementary to the counter-sequence, and thus preferably has the same length.

What is understood by an “isolated nucleic acid molecule” is a nucleic acid molecule extracted from its natural or original environment. The term also encompasses a synthetically-produced nucleic acid molecule. What is understood by an “isolated polypeptide” is a polypeptide extracted from its natural or original environment. The term also encompasses a synthetically-produced polypeptide.

A “molecular marker” is a nucleic acid that is polymorphic in a plant population and is used as a reference or orientation point. A marker for the detection of a recombination event should be suitable for monitoring differences or polymorphisms within a plant population. Such a marker is thus able to detect and differentiate between various allelic states (alleles). The term, “molecular marker,” also relates to nucleotide sequences which are complementary or at least largely complementary or homologous to genomic sequences—for example, nucleic acids which are used as probes or primers. These differences at the DNA level are to be found as markers and are, for example, polynucleotide sequence differences, e.g., SSR's (simple sequence repeats), RFLP's (restriction fragment length polymorphisms), FLP's (fragment length polymorphisms) or SNP's (single nucleotide polymorphisms). The markers may be derived from genomic or expressed nucleic acids, e.g., spliced RNA, cDNA, or EST's, and may also relate to nucleic acids that are used as probes or primer pairs and as such are suitable for amplifying a sequence fragment using PCR-based methods. Markers that describe genetic polymorphisms (between parts of a population) may be detected using well-established methods from the prior art (An Introduction to Genetic Analysis, 7th edition, Griffiths, Miller, Suzuki, et al., 2000). For example, among these are DNA sequencing, PCR-based, sequence-specific amplification, verification of RFLP's, verification of polynucleotide polymorphisms by means of allele-specific hybridization (ASH), detection of amplified variable sequences of the plant genome, detection of a 3SR (self-sustained sequence replication), detection of SSR's, SNP's, RFLP's, or AFLP's (amplified fragment length polymorphisms). Furthermore, the methods for detection of EST's (expressed sequence tags) and SSR markers derived from EST sequences and RAPD (randomly amplified polymorphic DNA) are also known. Depending upon the context, the term, “marker,” in the description may also mean a specific chromosome position in the genome of a species where a specific marker (SNP, for example) may be found.

Markers also include synthetic oligonucleotides that may be connected with one or more detection molecules, wherein the detection molecules may be used for a detection reaction or the generation of a signal within the scope of a verification method. Synthetic oligonucleotides also include labeled primers. Labeled primer include fluorescently labeled primers. Fluorescently labeled primers may be labeled by FAM or HEX. Synthetic oligonucleotides and labeled primers may by used within a polymerase chain reaction (PCR) as well as within a Kompetitive allele specific PCR. Synthetic oligonucleotides and labeled primers are artificial compounds, do not occur in nature, and cannot be isolated from nature. The production of such compounds is explained further below.

A “promoter” is a non-translated, regulatory DNA sequence, typically upstream of a coding region, which contains the binding point for the RNA polymerase and initiates the transcription of the DNA. A promoter additionally contains other elements that act as a regulator gene for gene expression (for example, cis-regulatory elements). A “core or minimal promoter” is a promoter that has the basic elements which are needed for transcription initiation (for example, TATA box and/or initiator).

A “pathogen” means an organism that, in interactions with a plant, leads to disease symptoms in one or more organs in the plant. For example, animal, fungal, bacterial, or viral organisms or oomycetes number among these pathogens.

What is to be understood by a “pathogenic infection” is the earliest point in time at which a pathogen interacts with a plant host tissue. In this sense, “infestation” means the occurrence of contact between pathogen and host. For example, the viral pathogen BNYVV is transmitted by the soil-borne fungus Polymyxa betae. Polymyxa forms spores which can survive in the soil for decades. The virus also survives in these spores. If these permanent spores lead to zoospores, the virus can germinate via these into cells of the plant host tissue and interact with the host (Esser (2000) Kryptogamen 1: Cyanobakterien Algen Pilze Flechten Praktikum and Lehrbuch. Springer-Verlag, Berlin, Heidelberg, 3rd edition).

Plant “organs” means, for example, leaves, shoot, stem, roots, hypocotyl, vegetative buds, meristems, embryos, anthers, ovula, seeds, or fruits. “Plant parts” include, but are not limited to, the shoot or the stalk, leaves, blossoms, inflorescence, roots, fruits, and seeds, as well as the pollen. The term, “plant parts,” also means an association of multiple organs, e.g., a blossom or a seed, or a part of an organ, e.g., a cross-section through the plant shoot. Plant “tissues” are, for example, callus tissue, storage tissue, meristematic tissue, leaf tissue, shoot tissue, root tissue, plant tumor tissue, or reproductive tissue, as well as the cambium, parenchyma, vascular tissue, sclerenchyma, and epidermis. However, the tissue is not limited to this listing. For example, what are to be understood by plant “cells” are, for example, isolated cells having a cell wall or aggregates thereof, or protoplasts.

The level of the resistance by way of example to BNYVV can be defined qualitatively in plants of the Beta genus by determination of “rating scores” (rating score schemes for plants of the Beta genus are known from the prior art, for example for sugar beet Mechelke (1997).

In conjunction with the present invention, the term, “regulatory sequence,” relates to a nucleotide sequence which influences the specificity and/or the expression strength, e.g., in that the regulatory sequence confers a defined tissue specificity. Such a regulatory sequence may be located upstream of the transcription initiation point of a minimal promoter, but also downstream thereof, e.g., in a transcribed, but not translated, leader sequence or within an intron.

The term, “resistance,” is to be understood broadly and covers the range of the protection from a retardation up to a complete blocking of the development of the disease. One example of an important pathogen is the Beet necrotic yellow vein virus (BNYVV). A resistant plant cell of the invention or resistant plant of the invention preferably achieves a resistance to BNYVV. A resistance to a pathogen is to be equated to a resistance to the disease which this pathogen causes; for example, a resistance to BNYVV is also a resistance to “root madness” (Rhizomania).

The “Rz2” resistance gene, which was identified and described in international patent application WO 2014/202044 A1 originates from the Beta maritima accession WB42 and is known to provide resistance to rhizomania when expressed in several varieties of sugar beet. Furthermore, it is known that Rz3 from the Beta maritima accession WB41 has been identified, which is most probably the same or a very similar allelic variant of Rz2. Thus, the Rz2 and Rz3 genes and respective proteins are substantially identical and the designation “Rz2” and “Rz3” can be used interchangeably.

“Remorins” are a family of plant-specific proteins containing a variable N-terminal region and conserved C-terminal domain and play a role in various biotic and abiotic stresses, including host-microbe interactions. The present invention distinguishes between a remorin gene, which encodes a functional remorin protein, i.e. a remorin protein which is involved in establishing Rz2 mediated BNYVV resistance in a plant in which the protein is expressed and a remorin gene which encodes a non-functional remorin protein, i.e. a remorin protein which interferes with Rz2 mediated BNYVV resistance in a plant in which the protein is expressed. In this context, the term “involved in” includes increasing the resistance as well as being essential for establishing the resistance against BNYVV in a plant additionally comprising the Rz2 resistance gene, in particular a high level of resistance against BNYVV. The term “non-functional” is used in terms of the inability to confer resistance in combination with the Rz2 gene.

Accordingly, the terms “functional form”, “functional (remorin) protein”, “functional (remorin) polypeptide”, “functional (remorin) variant”, “functional (remorin) allele”, “functional (remorin) gene”, and “functional nucleic acid molecule” of the present invention are used interchangeable in the present invention as well as the terms “non-functional form”, “non-functional (remorin) protein”, “non-functional (remorin) polypeptide”, “non-functional (remorin) variant”, “non-functional (remorin) allele”, “non-functional (remorin) gene, and “non-functional nucleic acid molecule” of the present invention.

“Transgenic plant” relates to a plant into whose genome is integrated at least one polynucleotide. It may thereby be a heterologous polynucleotide. The polynucleotide is, preferably, stably integrated, which means that the integrated polynucleotide is stably preserved in the plant, is expressed, and also may be stably passed on to the descendants. The stable introduction of a polynucleotide into the genome of a plant also includes the integration into the genome of a plant of the preceding parental generation, wherein the polynucleotide may be stably passed on further. The term, “heterologous,” means that the introduced polynucleotide originates from a cell or an organism with a different genetic background, of the same species or a different species, or is homologous to the prokaryotic or eukaryotic host cell, for example, but then is located in a different genetic environment and thus differs from a corresponding polynucleotide that is possibly naturally present. A heterologous polynucleotide may be present in addition to a corresponding endogenous gene.

“Raw material for industrial sugar production” means plant material which can be fed into a sugar production facility which is specialized in the extraction of sugar from sugar beets. Such raw material is typically the beet body (taproot) of the harvested sugar beet. To ensure the conformity with the extraction process the beet body needs to have sufficient mass, volume and a conical shape so that the raw material can be mechanically cut into shreds (beet strips). These beet strips maximize the surface area for sugar extraction and should have a low content of Sodium, Potassium and Nitrogen to allow an efficient extraction. After the extraction remaining beet pulp is pressed, dried and used as animal feed.

The “sucrose concentration” is expressed as percentage of the fresh weight of the root.

“Monogerm” means that a seed grows into exactly one plant whereas a polygerm or multigerm seed (also called “seed ball”) grows into several plants.

“Bolting” is the production of a flowering stem (or stems) on a sugar beet in a natural attempt to produce seeds and reproduce. Bolting is triggered in sugar beet due to vernalization, i.e. a chilling stress which might occur e.g. during overwintering. However, commercially grown sugar beets are harvested before bolting as the bolting process and subsequent seed setting reduces the sucrose content in the beet body.

“Kompetitive allele specific PCR” or “KASP” means a homogenous, fluorescence-based genotyping variant of polymerase chain reaction. It is based on allele-specific oligo extension and fluorescence resonance energy transfer for signal generation. This chemical reaction may be used for the detection of specific nucleic acid sequences in genomic DNA. Usually, fluorescently labeled primer are used for the PCR. FAM and HEX may be used as fluorescent tags.

Designs and embodiments of the present invention are described by way of example with reference to the pending sequences and figures.

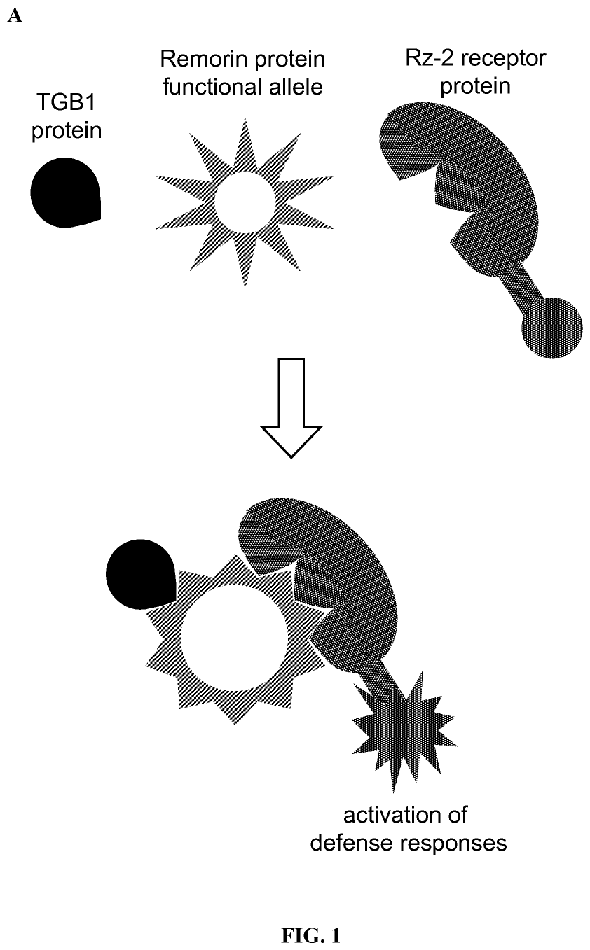

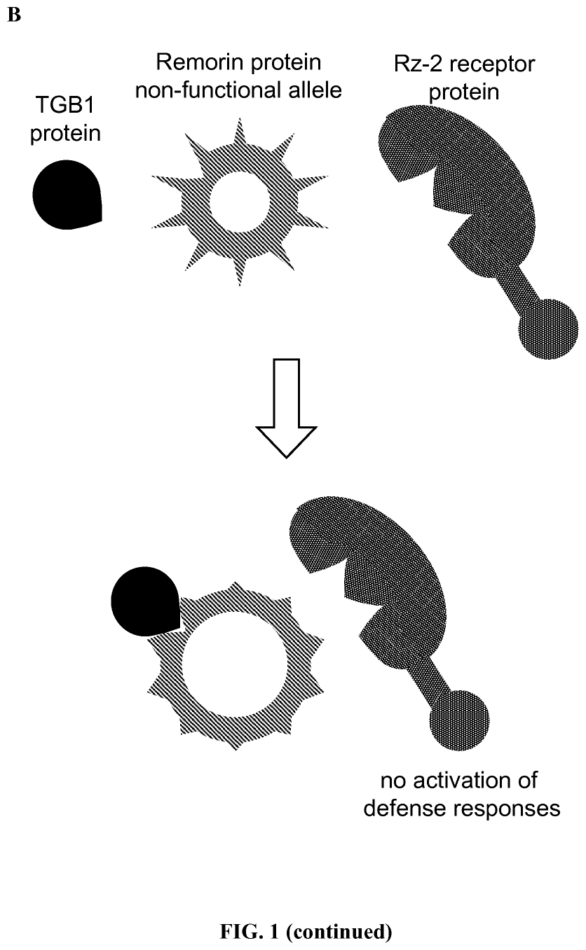

FIG. 1: Model depicting a possible mode of interaction between the TGB1 protein of Beet necrotic yellow vein virus (BNYVV), the Rz2 NBS-LRR receptor protein and (A) the functional variant of the remorin protein or (B) the non-functional variant of the remorin protein. In variant (A), the TGB1 protein binds to and modifies the functional remorin protein, which can be detected by the Rz2 NBS-LRR receptor protein of the resistance allele and thus, a resistance reaction is initiated. In variant (B), the Rz2 NBS-LRR receptor protein cannot detect the non-functional remorin protein modified by TGB1 and thus, no resistance reaction is initiated.

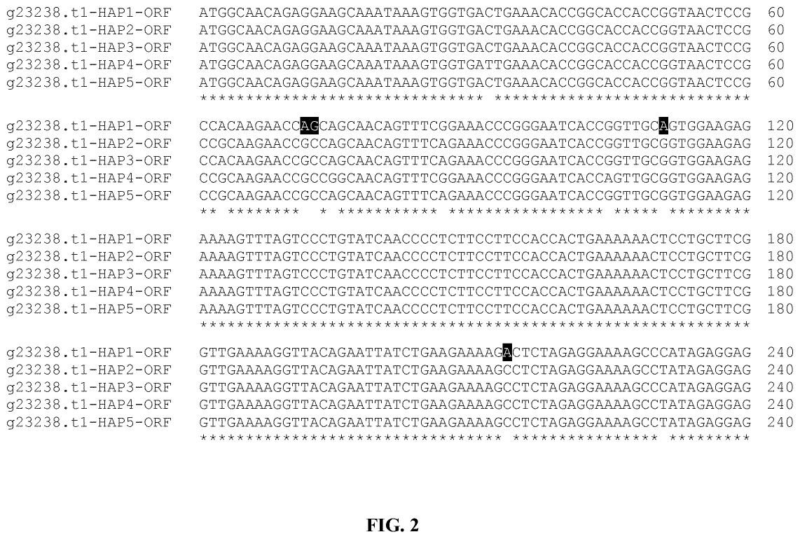

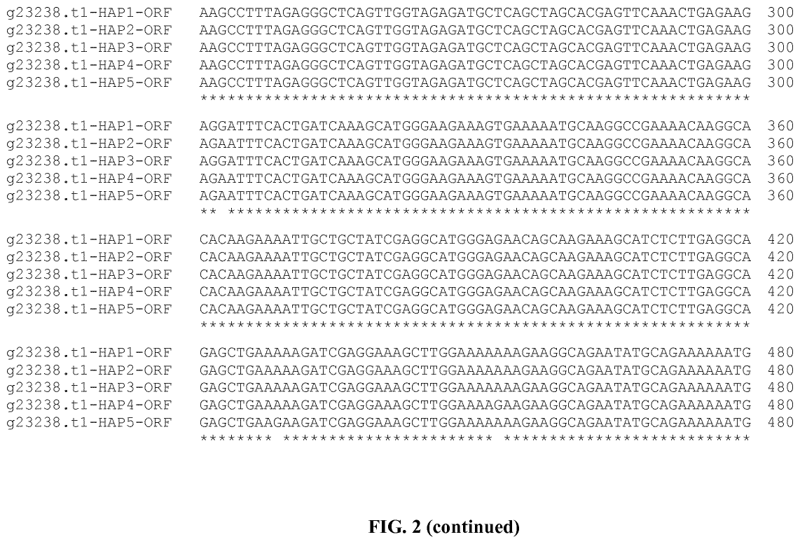

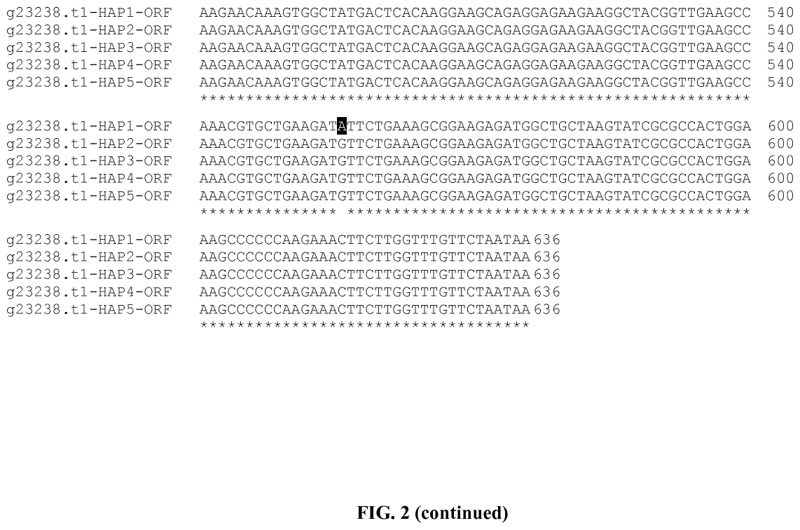

FIG. 2: Sequence alignment between the coding DNA (cDNA) sequence of the remorin gene encoding a non-functional remorin protein, i.e. a remorin protein which interferes with Rz2 mediated BNYVV resistance in a plant in which the protein is expressed (haplotype 1, Hap1-ORF), and the cDNA sequences of 4 haplotypes of the remorin gene encoding functional remorin proteins, i.e. remorin proteins which are involved in establishing Rz2 mediated BNYVV resistance in a plant in which the proteins are expressed (Hap2-ORF, Hap3-ORF, Hap4-ORF and Hap5-ORF). The polymorphisms are highlighted in gray.

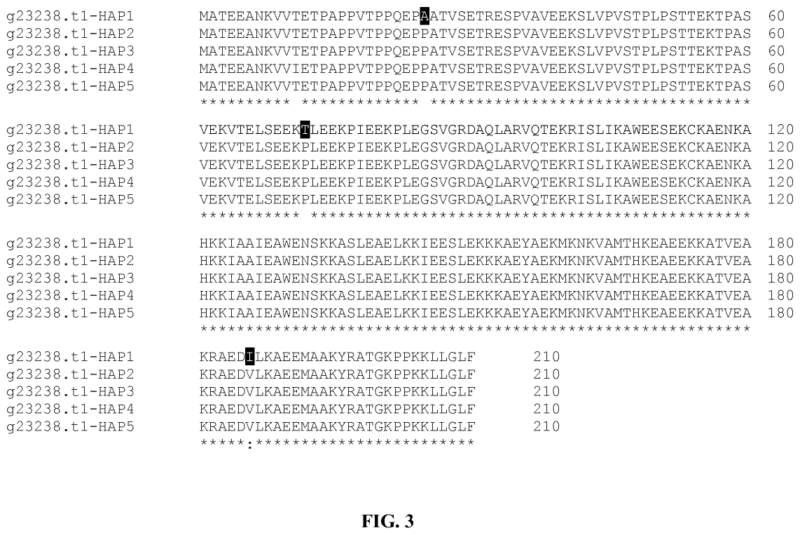

FIG. 3: Protein sequence alignment between a non-functional remorin protein, i.e. a remorin protein which interferes with Rz2 mediated BNYVV resistance in a plant in which the protein is expressed (Hap1), and 4 functional remorin proteins, i.e. remorin proteins which are involved in establishing Rz2 mediated BNYVV resistance in a plant in which the proteins are expressed (Hap2, Hap3, Hap4 and Hap5). The polymorphisms are highlighted in gray.

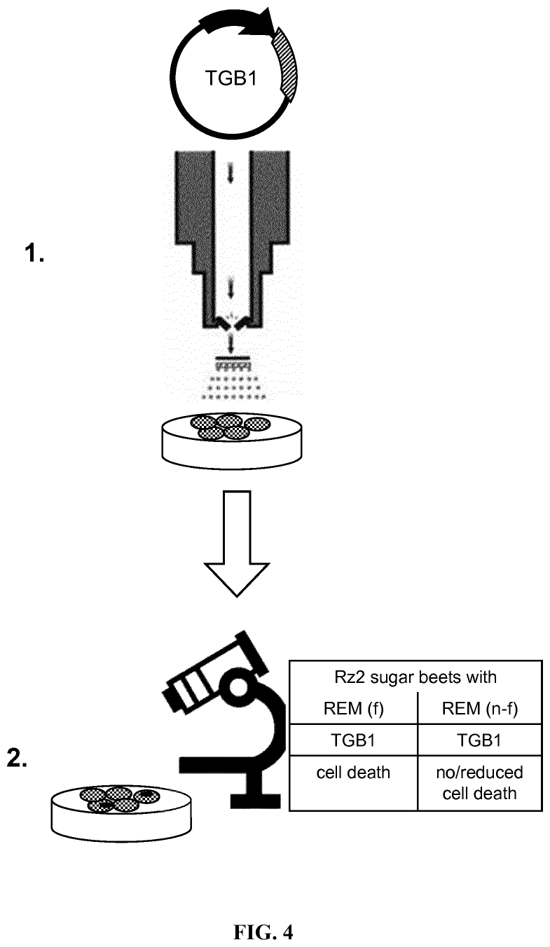

FIG. 4: Schematic overview of a transient assay for comparing the cell death reaction after expression of the TGB1 gene derived from BNYVV in leaf tissue of sugar beet comprising the resistance allele of Rz2 and either additionally comprising the functional allele of remorin (REM (f)) or the non-functional allele of remorin (REM (n-f)). The detailed experiment is explained in Example 2.

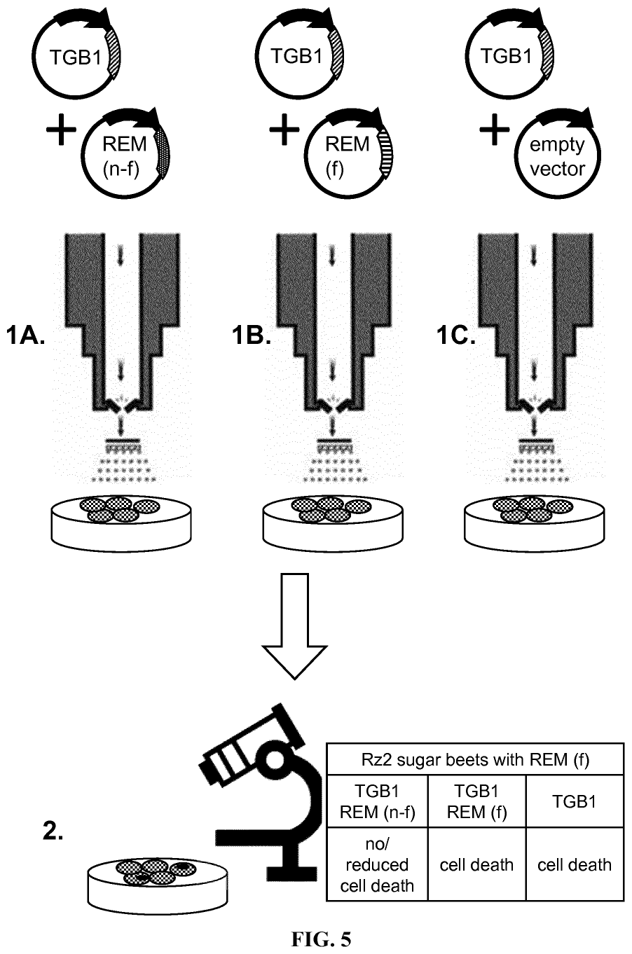

FIG. 5: Schematic overview of a transient assay for comparing the cell death reaction after expression of the TGB1 gene derived from BNYVV either in combination with the non-functional allele of remorin (REM (n-f)), with the functional allele of remorin (REM (f)), or with an empty vector control in leaf tissue of sugar beet comprising the resistance allele of Rz2 and the functional allele of remorin (REM (f)). The detailed experiment is explained in Example 2.

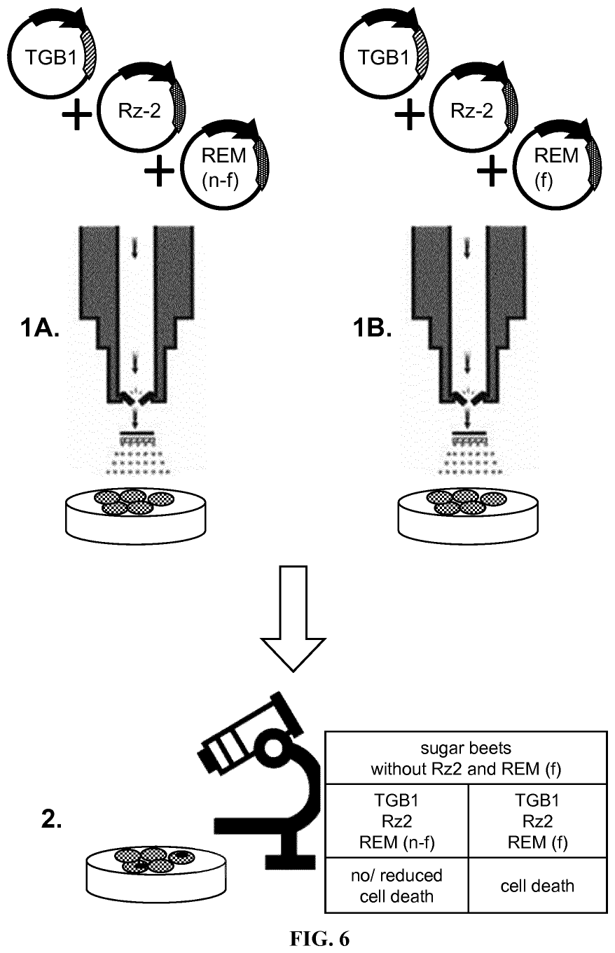

FIG. 6: Schematic overview of a transient assay for comparing the cell death reaction after expression of the TGB1 gene derived from BNYVV and the resistance allele of Rz2 either in combination with the non-functional allele of remorin (REM (n-f)) or with the functional allele of remorin (REM (f)) in leaf tissue of sugar beet which does neither comprise the resistance allele of Rz2 nor the functional allele of remorin (REM (f)). The detailed experiment is explained in Example 2.

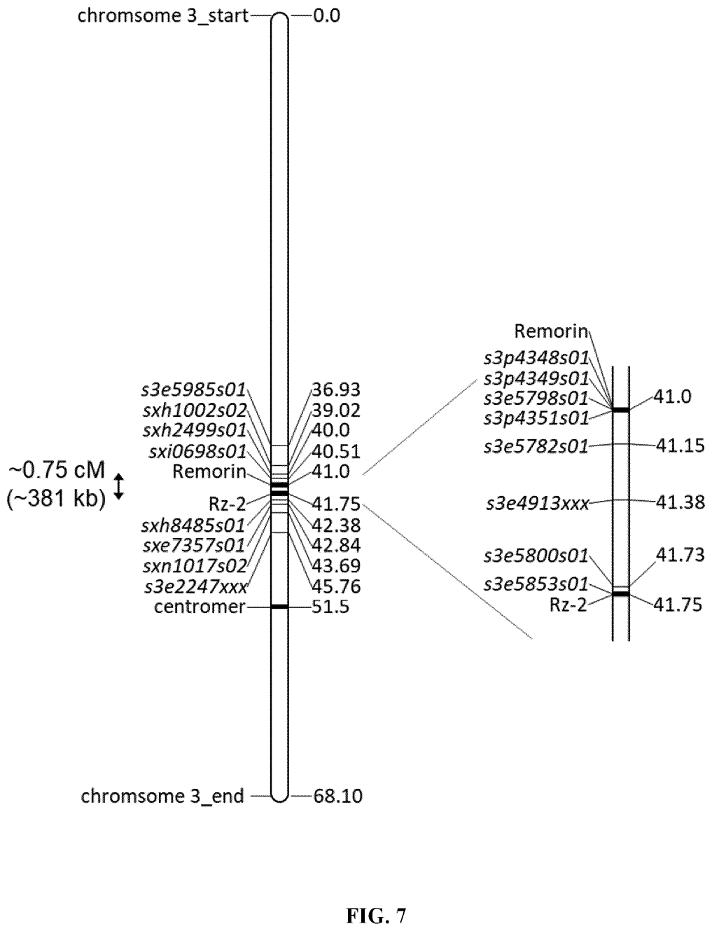

FIG. 7: Schematic view of chromosome 3 of Beta vulgaris indicating the positions of the remorin gene and the Rz2 gene as well as the positions of various markers. Remorin is positioned about 0.75 cM distal to the Rz2 gene on the long arm of chromosome 3. Left: marker positions distal to remorin and proximal to Rz2, which define flanking areas that can be used for marker assisted breeding (MAS); Right: markers binding in the remorin gene, in the Rz2 gene as well as in the region between both genes.

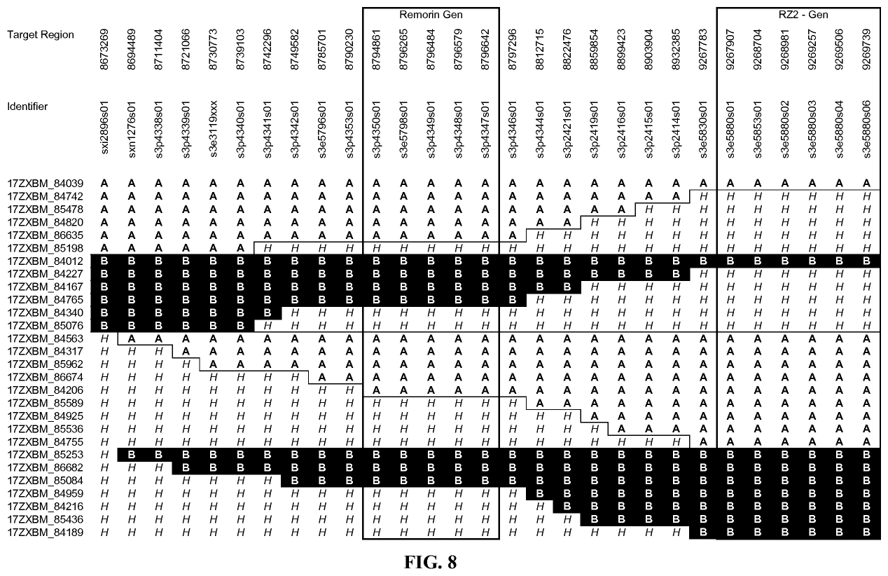

FIG. 8: Visual illustration of the marker-based analysis of 2730 plants of a dividing population with regard to the presence of different allele combinations of the Rz2 resistance gene and the remorin gene in the recombinants, wherein A indicates the presence of the remorin gene encoding a non-functional remorin protein and B indicates the presence of the remorin gene encoding a functional remorin protein. H designates the genotypes are heterozygous for the non-functional remorin allele.

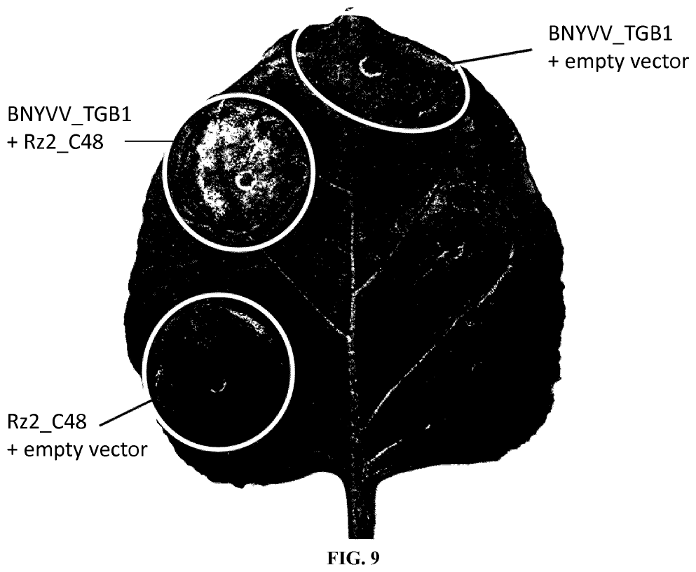

FIG. 9 The drawing shows a leaf of Nicotiana benthamiana after infiltration with Agrobacterium tumefaciens. The white surrounded spots mark the infiltrated areas wherein each area has been infiltrated with a different vector combination. The vector combinations are being explained in detail in the corresponding example.

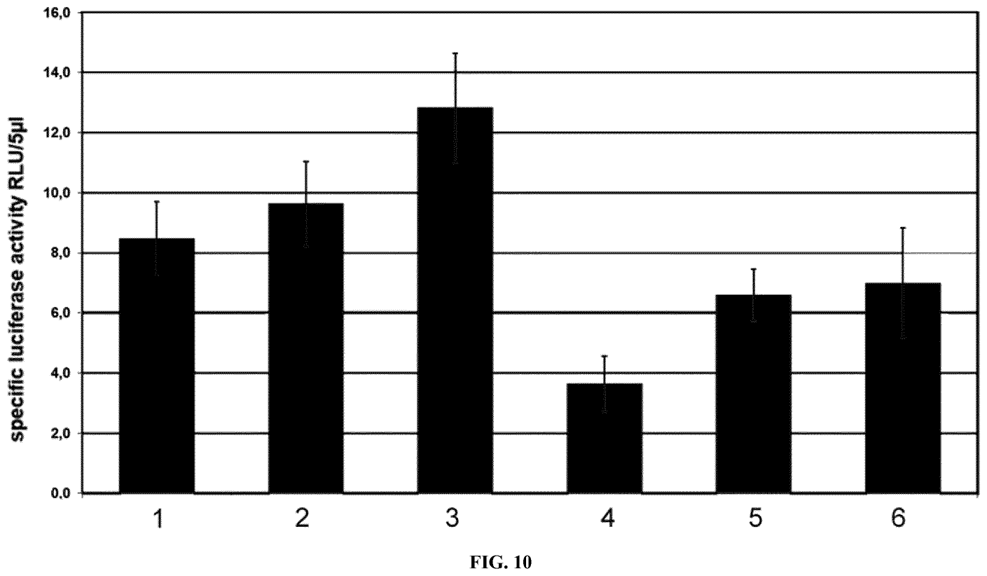

FIG. 10 Measurement of the luciferase activity in transformed leaf discs of sugar beet. Different constructs have been provided:
1: empty vector; 2: Rz2_C48+empty vector; 3: BNYVV_TGB1+empty vector; 4: Rz2_C48+BNYVV_TGB1; 5: Rz2_C48 D491V (autoactivated variant); 6: p70S-165-176_D80N (autoactivated CC domain, positive control)

### DETAILED DESCRIPTION OF THE INVENTION

In order to reveal the mechanism behind the observed effect that in certain sugar beet varieties the Rz2 mediated resistance against Beet necrotic yellow vein virus (BNYVV) is weakened or even completely lost, although the resistance allele of Rz2 is present and to use the gained knowledge for improving breeding processes, extensive crossing experiments as well as molecular biological experiments have been performed including genetic fine mapping, identification, isolation, and characterization of a gene encoding for a protein which in its functional form is important or crucial for establishing Rz2 mediated BNYVV resistance in plants and which in its non-functional form leads to suppression of said resistance.

The identification of both allelic variants, i.e. the one encoding a functional protein and the other encoding a non-functional protein is of high relevance in the breeding of BNYVV-resistant varieties that are to be equipped with Rz2 resistance. On the one hand, it is important to be able to separate the non-functional allele from the Rz2 resistance allele in order to use sugar beet lines showing a reduced or lost Rz2 mediated BNYVV resistance but having otherwise good breeding and agronomic properties in the breeding of BNYVV-resistant sugar beet varieties. On the other hand, it is of particular interest to understand the mechanism behind the development of Rz2 mediated resistance against BNYVV in plants, i.e. to identify the functional allele in order to efficiently generate plants being resistant to BNYVV.

Accordingly, the present invention generally relates to a nucleic acid molecule encoding a polypeptide which is involved in Rz2 mediated resistance against BNYVV. In this context, the term “involved in” includes increasing the resistance as well as being essential for establishing the resistance against BNYVV, in particular a high level of resistance against BNYVV. Thus, the nucleic acid molecule of the present invention and the encoded polypeptide, respectively, modifies the Rz2 mediated resistance against BNYVV in a plant in which the polypeptide is expressed. Preferably, the nucleic acid molecule of the present invention increases the resistance to BNYVV or is essential for establishing the resistance against BNYVV in a plant in which the corresponding polynucleotide is expressed. For the sake of simplicity, this nucleic acid molecule is referred to as “functional form”, “functional variant”, “functional allele”, “functional gene”, or “functional nucleic acid molecule” of the present invention.

Preferably, the nucleic acid molecule of the present invention modifies the Rz2 mediated resistance against BNYVV in a plant belonging to the genus Beta, preferably to the species Beta vulgaris, more preferably to the subspecies Beta vulgaris subsp. vulgaris.

The present invention further relates to a nucleic acid molecule which specifically hybridizes to the nucleic acid molecule of the present invention and encodes a non-functional allele of the polypeptide, i.e. a non-functional variant, which interferes with the resistance against BNYVV mediated by the Rz2 resistance gene in a plant in which the polypeptide is expressed. For the sake of simplicity, this nucleic acid molecule is referred to as “non-functional form”, “non-functional variant”, “non-functional allele”, “non-functional gene”, or “non-functional nucleic acid molecule” of the present invention.

In particular, the non-functional nucleic acid molecule of the present invention is able to reduce or suppress Rz2 mediated resistance to BNYVV in a plant in which the corresponding polynucleotide is expressed. Preferably said plant belongs to the genus Beta, preferably to the species Beta vulgaris, more preferably to the subspecies Beta vulgaris subsp. vulgaris.

The Rz2 resistance gene, which was previously identified and described in international patent application WO 2014/202044 A1 originates from the Beta maritima accession WB42 and is known to provide resistance to rhizomania when expressed in several varieties of sugar beet. The corresponding nucleic acid sequence is disclosed in WO 2014/202044 A1 and incorporated herein by reference. Rz3 from the Beta maritima accession WB41 has been identified, which is probably an allelic version of Rz2. Thus, the Rz2 and Rz3 genes are substantially identical and it is prudent to expect, but without to be bound by theory, that the nucleic acid molecule of the present invention is also involved in or interferes with Rz3 mediated resistance to BNYVV.

The identification and genetic localization of the nucleic acid molecule of the present invention has been performed as described in detail in Example 1. Initially, the focus was on the identification and localization of the non-functional allele. By genetic mapping it has been shown that the non-functional allele is localized close by the Rz2 locus. However, this area still contained too many genes to name a specific candidate. A particular challenge was the close genetic coupling/linkage of the non-functional allele with the Rz2 resistance gene. If it was located on another chromosome or at a greater distance from the Rz2 gene, the effect would be visible in QTL mapping as a separate effect. When using common breeding processes it was thus not possible to explain why genotypes were susceptible to rhizomania despite the presence of the resistance allele of Rz2.

Parallel to genetic mapping, the patterns of molecular markers in multigenic breeding material were analyzed. The marker s3e5798s01 (see Table 5) showed the highest evidence for the positioning of the repressor effect. Analysis of the reference sequence for this region showed that the marker s3e5798s01 is located in an annotated gene model (g23238.t1). The gene contains a remorin domain (C-terminal region) and is expressed in the plasmalemma. Remorins, a family of plant-specific proteins containing a variable N-terminal region and conserved C-terminal domain, play a role in various biotic and abiotic stresses, including host-microbe interactions. However, until now, their function is not fully understood.

There are indications in the literature that remorins can play a role in the spread of viruses through plasmodesmata (PDs) and in particular that they can act as negative regulator of virus movement (Raffaele et al., Plant Cell (2009), 21(5), 1541-1555; Perraki et al., FEMS Letters (2014), 588(9), 1699-1705). In general, PDs provide cell-to-cell connections that enable the symplastic transport of molecules from 1 to 60 kD. Viruses can move through PDs to achieve a local spreading infection, and symplastic loading into the phloem to initiate systemic infection.

In more detail, Raffaele et al. 2009 reported that in potato remorin interferes with cell-to-cell movement of Potato Virus X and binds directly to the virus movement protein TGBp1 (Triple Gene Block 1 protein). In plants underaccumulating remorin, a significant increase in viral accumulation in the leaves of infected plants has been observed and the infection foci were significantly larger than in wild-type plants. Consistently, reduced virus accumulation and smaller infection foci were observed in plants overexpressing remorin. Thus, remorin seems to have an antagonist effect on virus propagation. In this context, Perraki et al. 2014 have shown that in potatoes a remorin gene limits the ability of Potato Virus X TGBp1 to increase the permeability of PDs.

Furthermore, a recently published work by Wetzel and Varrelmann (International Plant Immunity Symposium—1st IRTG 2172 PRoTECT Symposium June 14th and 15th 2018, Gottingen, Germany) suggests that the protein produced by the Rz2 gene, i.e. the Rz2 NBS-LRR receptor protein, may interact with the TGBp1 protein of BNYVV, the virus causing rhizomania. The missing link between the Rz2 NBS-LRR receptor protein and the TGBp1 protein of BNYVV might be the remorin identified in accordance with the present invention. From this a possible mode of action can be derived, which is shown in FIG. 1A. Thus, it is to be assumed—but without being bound by one theory—that the remorin gene of the present invention has a similar or even the same function in Beta vulgaris as described above, i.e. that it acts as negative regulator of BNYVV movement.

The non-functional allele of the remorin gene identified in accordance with the present invention seems to be a non-functional protein. One possible mode of action is presented in FIG. 1B, wherein the Rz2 NBS-LRR receptor protein of the resistance allele cannot detect the modification of this allele by the TGB1 protein of BNYVV. Therefore, no resistance reaction is initiated.

Further experiments performed in accordance with the present invention confirmed this hypothesis in that first results showed that the combination of the resistance allele of Rz2 with the non-functional allele of remorin leads to plants still being susceptible to rhizomania, while plants comprising the resistance allele of Rz2 and the functional allele of remorin show resistance; see Example 2.

The knowledge gained throughout identification and analysis of the non-functional remorin allele has been used afterwards to identify the functional remorin allele. In particular, a comparative analysis of genomic sequences of a sugar beet variety comprising the non-functional remorin allele and thereby showing no or reduced BNYVV-resistance despite the presence of the Rz2 gene with sequences of 80 additional sugar beet genotypes has been performed in order identify the sequence of the functional remorin gene.

A total of five different haplotypes (alleles) were found. The haplotype of the non-functional remorin gene (haplotype 1) can be distinguished from the other four haplotypes, i.e. the functional remorin genes, by three KASP markers (s3p4348s01 (SEQ ID NO: 27; non-functional allele: G), s3p4349s01 (SEQ ID NO: 28; non-functional allele: T), s3p4351s01 (SEQ ID NO: 29; non-functional allele: T); see Table 5. The coding sequence, i.e. the open reading frame (ORF) of the non-functional allele set forth in SEQ ID NO: 3 has five small nucleotide polymorphisms (SNPs) which distinguish the haplotype (Hap1) from the other four haplotypes (Hap2-5; SEQ ID NOs: 7, 11, 15 and 19) (FIG. 2). Accordingly, the coding sequence (CDS) of Hap1 has an A instead of a G at position 72, a G instead of a C at position 73, an A instead of a G at position 111, an A instead of a C at position 214, and an A instead of a G at position 556; see Table 1. The protein sequence of the non-functional allele set forth in SEQ ID NO: 4 differs clearly in three amino acids from the other four haplotypes (SEQ ID NOs: 8, 12, 16 and 20); see Table 2. As shown in FIG. 3, the translated non-functional polypeptide encoded by the non-functional remorin gene has Ala instead of Pro at position 25, Thr instead of Pro at position 72 and Ile instead of Val at position 186; see also Table 2.

TABLE 1
Identified SNPs in coding sequences (ORFs) of the non-functional
remorin allele (= haplotype 1, Hap1) in comparison with the ORFs of
the other four haplotypes, i.e. the functional remorin alleles. Positions
marked with * lead to amino acid exchanges, which could be the
reason for the non-functionality of the remorin.

| CDS | Hap1 | Hap2 | Hap3 | Hap4 | Hap5 |
|---|---|---|---|---|---|
| nucleotide | (SEQ ID | (SEQ ID | (SEQ ID | (SEQ ID | (SEQ ID |
| position | No: 3) | No: 7) | No: 11) | No: 15) | No: 19) |
| 72 | A | G | G | G | G |
| 73* | G | C | C | C | C |
| 111 | A | G | G | G | G |
| 214* | A | C | C | C | C |
| 556* | A | G | G | G | G |

TABLE 2
Identified amino acid substitutions in the predicted sequence of the
polypeptide of the non-functional remorin allele (= haplotype 1) in
comparison with the predicted sequence of the polypeptide of
other four haplotypes, i.e. the functional alleles of remorin.

| amino | Hap1 | Hap2 | Hap3 | Hap4 | Hap5 |
|---|---|---|---|---|---|
| acid | (SEQ ID | (SEQ ID | (SEQ ID | (SEQ ID | (SEQ ID |
| position | No: 4) | No: 8) | No: 12) | No: 16) | No: 20) |
| 25 | Ala | Pro | Pro | Pro | Pro |
| 72 | Thr | Pro | Pro | Pro | Pro |
| 186 | Ile | Val | Val | Val | Val |

For the sake of simplicity, a consensus sequence has been generated combining the cDNA sequences of haplotypes 2 to 5 (SEQ ID No. 44) and another one combining the amino acid sequences of haplotypes 2 to 5 (SEQ ID No. 45).

Accordingly, in one embodiment the present invention relates to a nucleic acid molecule encoding a polypeptide which is involved in Rz2 mediated resistance against BNYVV, wherein the amino acid sequence of the encoded polypeptide has a proline (P) at position 25 with reference to SEQ ID No. 45, a proline (P) at position 72 with reference to SEQ ID No. 45 or a valine (V) at position 186 with reference to SEQ ID No. 45. Of course, the amino acid sequence of the encoded polypeptide may have two of the mentioned amino acids at the indicated positions, i.e. a proline (P) at position 25 and a proline (P) at position 72 with reference to SEQ ID No. 45; a proline (P) at position 25 and a valine (V) at position 186 with reference to SEQ ID No. 45; or a proline (P) at position 72 and a valine (V) at position 186 with reference to SEQ ID No. 45; as well as three of the mentioned amino acids at the indicated positons, i.e. a proline (P) at position 25, a proline (P) at position 72 and a valine (V) at position 186 with reference to SEQ ID No. 45.

With reference to the coding DNA sequence of said nucleic acid molecule, the coding DNA sequence has a cytosine (c) at position 73 with reference to SEQ ID No. 44, a cytosine (c) at position 214 with reference to SEQ ID No. 44, or a guanine (g) at position 556 with reference to SEQ ID No. 44. Of course, the coding DNA sequence may have two of the mentioned amino acids at the indicated positons, i.e. a cytosine (c) at position 73 and a cytosine (c) at position 214 with reference to SEQ ID No. 44; a cytosine (c) at position 73 and a guanine (g) at position 556 with reference to SEQ ID No. 44; or a cytosine (c) at position 214 and a guanine (g) at position 556 with reference to SEQ ID No. 44; as well as three of the mentioned amino acids at the indicated positons, i.e. a cytosine (c) at position 73, a cytosine (c) at position 214 and a guanine (g) at position 556 with reference to SEQ ID No. 44.

In a preferred embodiment, the present invention relates to a nucleic acid molecule encoding a polypeptide which is involved in Rz2 mediated resistance against BNYVV, wherein the polypeptide has the amino acid sequence of SEQ ID No. 8, 12, 16, or 20, or the nucleotide sequence that encodes said polypeptide is SEQ ID No. 5, 6, 7, 9, 10, 11, 13, 14, 15, 17, 18 or 19, or the coding DNA sequence is a nucleotide sequence according to SEQ ID No. 7, 11, 15, or 19.

Furthermore, substitutions, deletions, insertions, additions, and/or any other change may be introduced into the nucleotide sequence according to the invention that, alone or in combinations, do in fact change the nucleotide sequence, wherein the modified nucleotide sequence may, however, perform the same function as the initial sequence. The present case deals with the coding of an amino acid sequence which is involved in resistance against BNYVV mediated by the Rz2 resistance gene.

In a further embodiment, the invention therefore includes a nucleotide sequence that encodes a polypeptide which represents a derivative of the polypeptide which is encoded by the nucleotide sequence according to the invention, or which includes the amino acid sequence according to the invention. A derived amino acid sequence which has at least one substitution, deletion, insertion, or addition of one or more amino acids, wherein the functionality of the encoded polypeptide/protein is preserved, represents a derivative of the polypeptide. Substitutions, deletions, insertions, additions, and/or any other change, either solely or in combinations, that do in fact change the nucleotide sequence, but perform the same function as the initial sequence, may thereby be introduced into the nucleotide sequence using conventional methods that are known in the prior art, e.g., via site-directed mutagenesis, PCR-mediated mutagenesis, transposon mutagenesis, genome editing, etc.

The substitution of one amino acid by a different amino acid having the same or equivalent or similar chemical/physical properties is referred to as a “conservative substitution” or “semi-conservative substitution.” Examples of physical/chemical properties of an amino acid are, for example, hydrophobia or the charge. Which amino acid substitution represents a conservative or semi-conservative substitution is known to the person skilled in the art. Moreover, general expertise allows the person skilled in the art to recognize, identify, and detect which amino acid deletions and additions are harmless to the functionality of the resistance protein, and at which positions these are possible. The person skilled in the art is aware that, in the case of the present functional remorin protein for modifications of the amino acid sequence (substitutions, deletions, insertion, or additions of one or more amino acids), the functionality, in particular, of the conserved domains, e.g. the remorin domain must be preserved, and that therefore only limited preceding modifications are possible in these domains.

The invention thus includes a functional fragment of the nucleotide sequence according to the invention. The term, “fragment,” thereby includes genes with a nucleotide sequence sufficiently similar to the aforementioned nucleotide sequence. The term, “sufficiently similar,” means that a first nucleotide sequence or amino acid sequence has a sufficient or minimum number of identical or equivalent nucleotides or amino acid groups relative to a second nucleotide sequence or a second amino acid sequence.

With regard to the amino acid sequence, after modification via an aforementioned method, this also has a common structural domain and/or possesses common functional activity. Nucleotide sequences or amino acid sequences that have an identity of at least approximately 70%, at least approximately 75%, at least approximately 80%, at least approximately 85%, at least approximately 90%, at least approximately 91%, at least approximately 92%, at least approximately 93%, at least approximately 94%, at least approximately 95%, at least approximately 96%, at least approximately 97%, at least approximately 98%, at least approximately 99%, or at least approximately 100% with the nucleotide sequence or amino acid sequence according to the invention are defined here as being sufficiently similar. This also explicitly encompasses the range of 90% to 100%. For the functional fragments, a sufficient similarity is established if the nucleotide sequence or amino acid sequence generally has the same property as the previously-named nucleotide sequence or amino acid sequence of the present invention. Those nucleotide sequences which encode a derivative or for a derived amino acid sequence are generated either directly or indirectly (for example, via amplification or replication steps) from an initial nucleotide sequence which corresponds to the nucleotide sequence according to the invention over the entire length, or at least in part.

Accordingly, the present invention includes a nucleotide sequence that is able to hybridize, under stringent conditions, with a nucleotide sequence complementary to a nucleotide sequence according to the invention or to the nucleotide sequence that encodes the amino acid sequence according to the invention.

The present invention further relates to a nucleic acid molecule which specifically hybridizes to the nucleic acid molecule of the present invention and encodes a non-functional allele of the polypeptide, i.e. a non-functional remorin variant, which interferes with resistance against BNYVV mediated by the Rz2 resistance gene in a plant in which the polypeptide is expressed. For the sake of simplicity, this nucleic acid molecule is referred to as “non-functional form”, “non-functional variant”, “non-functional allele”, or “non-functional gene”, or “non-functional nucleic acid molecule” of the present invention.

As shown in Example 2, in particular in Assay 2, the non-functional polypeptide (remorin) allele seems to be dominant over the functional allele of the polypeptide. Accordingly, in a preferred embodiment of the present invention, the non-functional polypeptide encoded by the nucleic acid molecule according to [3] is dominant over the functional allele of the polypeptide encoded by a nucleic acid molecule according to [1] or [2]. Dominant means that in the heterozygous state of the plant, e.g., the plant comprising a non-functional allele and a functional allele of the polypeptide the non-functional allele inhibits or prevents the development of resistance and as a result the plant is susceptible to BNYVV infection and spread of the virus within the plant, respectively.

In particular, the non-functional nucleic acid molecule of the present invention is able to reduce or suppress Rz2 mediated resistance to BNYVV in a plant in which the corresponding polynucleotide is expressed. Preferably said plant belongs to the genus Beta, preferably to the species Beta vulgaris, more preferably to the subspecies Beta vulgaris subsp. vulgaris.

The nucleotide and amino acid encoding sequence of said non-functional allele is characterized by polymorphisms, which differentiates it from the functional allele, i.e. the nucleic acid molecule of the present invention which modifies the Rz2 mediated resistance against BNYVV in a plant in which the polypeptide is expressed. In principle, the sequence of the non-functional gene of the present invention and the corresponding polypeptide, respectively is based on the sequence of the nucleic acid molecule of the present invention as described above but including one or more mutations as already defined above leading to a non-functional variant. There are of course plenty of possibilities for mutations leading to the non-functional variant and the person skilled in the art of course knows how to introduce single mutations and how to test whether the generated gene and polypeptide, respectively is indeed inactive. Details regarding suitable methods are described elsewhere herein.

In a preferred embodiment, the non-functional nucleic acid molecule of the present invention comprises a nucleotide sequence encoding a polypeptide having an amino acid sequence according to SEQ ID No. 4 or a polypeptide that is of at least approximately 70%, at least approximately 75%, at least approximately 80%, at least approximately 85%, at least approximately 90%, at least approximately 91%, at least approximately 92%, at least approximately 93%, at least approximately 94%, at least approximately 95%, at least approximately 96%, at least approximately 97%, at least approximately 98%, at least 99% or 100% identical to SEQ ID No. 4, wherein the polypeptide comprises an amino acid sequence having at least one amino acid substitution due to one or more mutations in the nucleotide sequence, preferably wherein proline (P) at position 25 with reference to SEQ ID No. 45, proline (P) at position 72 with reference to SEQ ID No. 45, and/or valine (V) at position 186 with reference to SEQ ID No. 45 is substituted with another amino acid. Of course, the amino acid sequence may have one, two or three amino acid substitutions at the indicated positions. Thus, in one embodiment, proline (P) at position 25, proline (P) at position 72, or valine (V) at position 186 with reference to SEQ ID No. 45 is substituted with another amino acid. In a further embodiment, proline (P) at position 25 and proline (P) at position 72 with reference to SEQ ID No. 45 are substituted with another amino acid; or proline (P) at position 25 and valine (V) at position 186 with reference to SEQ ID No. 45 are substituted with another amino acid; or proline (P) at position 72 and valine (V) at position 186 with reference to SEQ ID No. 45 are substituted with another amino acid. Alternatively, proline (P) at position 25, proline (P) at position 72 and valine (V) at position 186 with reference to SEQ ID No. 45 are substituted with another amino acid.

In a preferred embodiment, the non-functional remorin polypeptide of the present invention comprises an amino acid sequence having alanine (A) instead of proline (P) at position 25 with reference to SEQ ID No. 45, threonine (T) instead of proline (P) at position 72 with reference to SEQ ID No. 45, and/or isoleucine (I) instead of valine (V) at position 186 with reference to SEQ ID No. 45. Of course, the amino acid sequence may have one, two or all three amino acid substitutions at the indicated positions as explained above.

With respect to the coding DNA sequence, in a preferred embodiment, the non-functional remorin gene of the present invention comprises a nucleotide sequence that comprises the coding DNA sequence according to SEQ ID No. 3, that comprises a coding DNA sequence which is at least 70% identical to the DNA sequence according to SEQ ID No. 3, or that hybridizes with a complementary sequence of SEQ ID No. 3 under stringent conditions, wherein the nucleotide sequence comprises at least one nucleic acid substitution due to at least one mutation leading to an amino acid substitution, preferably wherein one or more nucleotides are substituted at positions 73-75 with reference to SEQ ID No. 44, at positions 214-216 with reference to SEQ ID No. 44 and/or at positions 556-558 with reference to SEQ ID No. 44. Of course, the coding DNA sequence may have substituted nucleotides at one, two or all three positions indicated. Thus, in one embodiment, one or more nucleotides are substituted at positions 73-75, at positions 214-216, or at positions 556-558 with reference to SEQ ID No. 44.

In a further embodiment, one or more nucleotides are substituted at positions 73-75 and at positions 214-216 with reference to SEQ ID No. 44; or one or more nucleotides are substituted at positions 73-75 and at positions 556-558 with reference to SEQ ID No. 44; or one or more nucleotides are substituted at positions 214-216 and at positions 556-558 with reference to SEQ ID No. 44. Alternatively, one or more nucleotides are substituted at positions 73-75, at positions 214-216, and at positions 556-558 with reference to SEQ ID No. 44.

In a preferred embodiment, the coding DNA sequence has guanine (g) instead of cytosine (c) at position 73 with reference to SEQ ID No. 44, adenine (a) instead of cytosine (c) at position 214 with reference to SEQ ID No. 44, and/or adenine (a) instead of guanine (g) at position 556 with reference to SEQ ID No. 44. Of course as mentioned above, the coding DNA sequence may have substituted nucleotides at one, two or all three positions indicated.

Optionally, the coding DNA sequence has in addition adenine (a) instead of guanine (g) at that position 72 with reference to SEQ ID No. 44, and/or adenine (a) instead of guanine (g) at position 111 with reference to SEQ ID No. 44.

However, as described above with respect to the functional remorin allele, the person skilled in the art of course knows that further substitutions, deletions, insertions, additions, and/or any other change may be introduced that, either alone or in combinations, do in fact change the nucleotide sequence, but perform the same function as the initial sequence—here, the nucleotide sequence encoding the non-functional variant of the remorin gene.

The nucleic acid molecule according to the invention, including the nucleic acid molecule which is involved in conferring a resistance against the pathogen BNYVV and the corresponding non-functional variant interfering with said resistance, may be an isolated nucleic acid molecule. It is preferably DNA, and, particularly preferably, cDNA (coding DNA). The functional remorin protein is involved in conferring a resistance against the pathogen BNYVV which causes the plant disease rhizomania, more preferably it modifies the Rz2 mediated resistance against BNYVV in a plant in which the protein is expressed, i.e. which increases said resistance to BNYVV or is essential for establishing said resistance. The non-functional remorin protein interferes with said resistance. Furthermore, the polypeptide is involved in conferring a resistance to this pathogen and interferes with said resistance, respectively, in particular in a plant of the genus Beta. The plant is preferably a plant of the species Beta vulgaris—particularly preferably, a plant of the subspecies Beta vulgaris subsp. vulgaris; among these are, for example, the cultivars sugar beet, beetroot, fodder beet, chard, and Swiss chard.

When not stated otherwise, if referred to the nucleic acid molecule or the polypeptide of the present invention, the nucleic acid molecule and the polypeptide, respectively which is involved in Rz2 mediated resistance against BNYVV, i.e. the function remorin gene and the functional remorin polypeptide/protein, as well as the nucleic acid molecule and the polypeptide, respectively which interferes with the Rz2 mediated resistance against BNYVV, i.e. the non-function remorin gene and the non-functional remorin polypeptide/protein, are included.

Such a gene that modifies the Rz2 mediated resistance, or that encodes a polypeptide that is able to modify the Rz2 mediated resistance is not known from the prior art. As was already described above, in previously available varieties on the market, it has been observed that certain genetic backgrounds led to a weakening or loss of resistance against BNYVV, although the resistant allele of Rz2 was present the corresponding plants. With the identification of the gene being responsible for this effect, i.e. the non-functional variant of the remorin gene, which allows the generation of suitable markers, it is now possible to diagnose the non-functional remorin allele due to its unique haplotype. Furthermore, the exact knowledge of the genetic and physical positions of the Rz2 gene and the remorin gene allows for the first time to identify recombinants between the genes and thus to separate the non-functional remorin gene from the Rz2 resistance allele. In new crossings with markers located on or between remorin and Rz2, recombinants showing the functional allele of the crossing partner can be selected. Furthermore, the identification of the functional remorin allele allows the incorporation of this gene into plants, e.g. into high-yield varieties, having the Rz2 resistance gene but being no longer resistant to BNYVV due to the presence of the non-functional variant of remorin.

Thus, via the incorporation of the nucleic acid molecule of the present invention in said plants, i.e. the function remorin gene, optionally in combination with removing the non-functional remorin gene, e.g. via inhibiting its expression at all, or via mutating the non-functional remorin gene in order to establish the functional variant, it is now possible to very quickly develop high-yield varieties with a restored resistance to BNYVV. Accordingly, in the framework of the present invention there are provided for the first time a sugar beet plant, a chard plant, a red beet or beetroot plant, a fodder beet plant having the resistance mediated by the Rz2 gene and the functional remorin gene according to the invention against BNYVV and thus being encompassed by the present invention. As the listed plants are all cultivated plants, crops or plants which are suitable for the agricultural cultivation and which have the resistance according to the invention, are part of the invention. Especially such crops are part of the invention which comprise a subterrestrial storage organ usable as food, raw material or industrial source of sugar and which comprise the resistance according to the invention are a further aspect of the present invention. The storage organ can be for example the sugar containing beet body of the sugar beet, the consumable beet body of the red beet or the feedable beet body of the fodder beet. The subterrestrial storage organ can sum up to more than 50% and for the sugar beet even to more than 70% of the total mass of the full-grown plant. Furthermore, also seeds or seeding material of these plants are part of the invention. The seeds or the seeding material can be technically treated as described further below.

Furthermore, the present invention relates to a recombinant and/or heterologous DNA molecule that comprises the sequences of the nucleic acid molecule according to the invention, i.e. the nucleic acid molecule involved in Rz2 mediated resistance to BNYVV and the nucleic acid molecule which interferes with said resistance, respectively. This DNA molecule, furthermore, preferably has a regulatory sequence. It may thereby be operatively linked with this regulatory sequence or be under the influence of this regulatory sequence. This regulatory sequence is preferably a promoter sequence and/or other sequences of transcription or translation control elements—for example, cis-elements. The regulatory sequence, which controls the expression of a gene that includes the nucleic acid molecule according to the invention, is preferably a sequence that is able to confer or modulate the expression, as a result of a pathogenic infection. This promoter is preferably able to control the expression of the DNA sequence specifically in roots of the plant. The regulatory sequence may be heterologous to the expressing sequence. Such an approach has the advantage that the person skilled in the art may better adjust the expression rate of the expressing sequence, the tissue in which the expression occurs, and the point in time at which the expression occurs, in that he selects that regulatory sequence which is best suited to the respective use case. The heterologous DNA sequence preferably includes a nucleotide sequence which encodes a component of the plant pathogen defense (example: resistance genes (R-genes) or genes which encode enzymes involved in signal transfer, such as kinases or phosphatases, and for G-protein, or which encode a pathogenic effector (what are known as avirulence genes (avr))). The heterologous DNA sequence may be one of the DNA sequences according to the invention. The heterologous DNA sequence may also additionally encode further components of the plant pathogen defense. The heterologous DNA sequence may therefore be designed such that a polycistronic mRNA is created after its transcription.

The present invention further relates to a polypeptide which can be encoded by the nucleic acid molecule according to the invention and a functionally and/or immunologically active fragment thereof, as well as an antibody that specifically binds to the polypeptide or to its fragment. The polypeptide particularly preferably has an amino acid sequence according to any one of SEQ ID Nos. 4, 8, 12, 16 and 20. The recombinant production of proteins, polypeptides, and fragments is familiar to the person skilled in the art (Sambrook et al., Molecular Cloning: A Laboratory Manual, 3rd ed., Cold Spring Harbor Laboratory Press, Cold Spring Harbor, N.Y., 2001, or Wingfield, P. T., 2008, Production of Recombinant Proteins, Current Protocols in Protein Science, 52:5.0:5.0.1-5.0.4). Polyclonal or monoclonal antibodies to the protein according to the invention may be produced by the person skilled in the art according to known methods (E. Harlow et al., editor, Antibodies: A Laboratory Manual (1988)). The production of monoclonal antibodies, as well as of Fab and F(ab′)2 fragments that are also useful in protein detection methods, may be performed via various conventional methods (Goding, Monoclonal Antibodies: Principles and Practice, pp. 98-118, New York: Academic Press (1983)). The antibodies may then be used for the screening of expression cDNA libraries in order to identify identical, homologous, or heterologous genes by means of immunological screening (Sambrook et al., Molecular Cloning: A Laboratory Manual, Cold Spring Harbor Laboratory Press, Cold Spring Harbor, N.Y., 1989, or Ausubel et al., 1994, “Current Protocols in Molecular Biology.” John Wiley & Sons), or may be used for western blot analyses.

In particular, the present invention relates to antibodies that selectively detect a polypeptide encoded by the nucleic acid molecule of the present invention involved in Rz2 mediated resistance to BNYVV, i.e. the functional remorin allele and essentially do not detect the polypeptide encoded by the nucleic acid molecule of the present invention which interferes with said resistance, i.e. the non-functional remorin allele. In particular, they detect less, by a factor of 2—preferably, a factor of 5, and, more preferably, a factor or 10 or more—of the polypeptide encoded by the correspondingly non-functional remorin allele than the polypeptide encoded by the functional remorin allele. In another embodiment, the present invention relates to antibodies that selectively detect a polypeptide encoded by the nucleic acid molecule of the present invention which interferes with the Rz2 mediated resistance to BNYVV, i.e. the non-functional remorin allele and essentially do not detect the polypeptide encoded by the nucleic acid molecule of the present invention which is involved in said resistance, i.e. the functional remorin allele. In particular, they detect less, by a factor of 2—preferably, a factor of 5, and, more preferably, a factor or 10 or more—of the polypeptide encoded by the correspondingly functional remorin allele than the polypeptide encoded by the non-functional remorin allele.

In a preferred embodiment, the antibody according to the invention is characterized in that it is a synthetic polypeptide which does not occur in nature.

Furthermore, the antibodies according to the invention may be linked with a fluorescent dye in order to be usable in an immunohistochemical method, for example, and evoke an antibody coloration. The fluorescent dye may be fluorochrome. The antibodies according to the invention may also be present linked with other signaling molecules. Among these are, for example, biotin, radioisotopes, reporter enzymes such as alkaline phosphatase, or oligonucleotides.

An additional subject matter of the invention are vectors or expression cassettes that include the nucleic acid molecule or the recombinant DNA molecule according to the invention—possibly under control of regulatory elements and, in particular, under control of functional regulatory elements in plants, as well as negative and/or positive selection markers. The vector backbone is thereby heterologous to the nucleic acid molecule according to the invention, which means that such a vector does not occur in nature and cannot be isolated from nature. The vector is a plasmid, a cosmid, a phage or an expression vector, a transformation vector, shuttle vector or cloning vector; it may be double-stranded or single-stranded, linear or circular; or it may transform a prokaryotic or eukaryotic organism either via integration into its genome or extrachromosomally. The nucleic acid molecule or DNA molecule according to the invention in an expression vector or expression cassette is, preferably, operatively linked with one or more regulatory sequences which allow the transcription and, optionally, the expression in a prokaryotic or eukaryotic cell; (Sambrook et al., Molecular Cloning: A Laboratory Manual, 3rd ed., Cold Spring Harbor Laboratory Press, Cold Spring Harbor, N.Y., 2001). These regulatory sequences are preferably promoters or terminators—in particular, a transcription initiation starting point, a ribosome binding location, an RNA-processing signal, a transcription termination location, and/or a polyadenylation signal. For example, the nucleic acid molecule is here under the control of a suitable promoter and/or a terminator. Suitable promotors may be constitutive promotors (example: 35S promoter from the “Cauliflower mosaic virus” (Odell et al., Nature 313 (1985), 810-812); those promoters which are pathogenically inducible are especially suitable (example: PR1 promoter from parsley (Rushton et al., EMBO J. 15 (1996), 5,690-5,700)). Particularly suitable pathogenically-inducible promoters are synthetic or chimeric promoters which do not occur in nature, are composed of multiple elements, and contain a minimal promoter, and have at least one cis-regulatory element upstream of the minimal promoter, which at least one cis-regulatory element serves as a binding location for special transcription factors. Chimeric promoters are designed according to the desired requirements and are induced or repressed via different factors. Examples of such promoters are found in WO 00/29592, WO 2007/147395, and WO 2013/091612. For example, a suitable terminator is the nos-terminator (Depicker et al., J. Mol. Appl. Genet. 1 (1982), 561-573).

Suitable promoters and terminators may also be the native promoter and the native terminator. The vectors or expression cassettes additionally contain for conventional indicator/reporter genes or resistance genes for the detection of the transfer of the desired vector or DNA molecule/nucleic acid molecule, and for selection of the individuals that contain these, since a direct detection via the expression of the gene is for the most part rather difficult.

Examples of indicator/reporter genes are, for example, the luciferase gene and the gene encoding green fluorescent protein (GFP). These, furthermore, also allow tests for the activity and/or regulation of a promoter of the gene. Examples of resistance genes—especially, for plant transformations—are the neomycin phosphotransferase gene, the hygromycin phosphotransferase gene, or the gene encoding phosphinothricin acetyltransferase. Additional positive selection markers may be enzymes which provide the transformed plant a selection advantage over the non-transformed plant—in particular, a nutrient advantage, e.g., the mannose-6-phosphate isomerase or the xylose isomerase. However, this does not preclude additional indicator/reporter genes or resistance genes known to the person skilled in the art. In a preferred embodiment, the vector is a plant vector. Furthermore, the expression cassette may be present as integrated into a plant genome.

In a further aspect, the present invention relates to cells that include the vectors, recombinant DNA molecules, and/or nucleic acid molecules according to the invention. A cell in the sense of the invention may be a prokaryotic (for example, bacterial) or eukaryotic cell (for example, a plant cell or a yeast cell). The cell is preferably an agrobacterium such as Agrobacterium tumefaciens or Agrobacterium rhizogenes, an Escherichia coli cell, or a plant cell; the plant cell is particularly preferably a cell of a plant of the genus Beta, the species Beta vulgaris, or the subspecies Beta vulgaris subsp. vulgaris. The cell may also be present as a culture. The invention also consequently covers a cell culture which contains such cells. The cell culture is preferably a pure culture or an isolate that contains no cells of another type.

Known to the person skilled in the art are both numerous methods, such as conjugation or electroporation, with which he may introduce the nucleic acid molecule according to the invention, the recombinant DNA molecule, and/or the vector or the expression cassette of the present invention into an agrobacterium, and methods such as diverse transformation methods (biolistic transformation, agrobacterium-mediated transformation) with which he may introduce the nucleic acid molecule according to the invention, the DNA molecule, and/or the vector of the present invention into a plant cell (Sambrook et al., Molecular Cloning: A Laboratory Manual, 3rd ed., Cold Spring Harbor Laboratory Press, Cold Spring Harbor, N.Y., 2001.

Furthermore, the present invention relates to a plant—preferably, plant of the species Beta vulgaris subsp. vulgaris or a portion thereof—that contains the nucleic acid molecule according to the invention which is involved in resistance against BNYVV, i.e. the functional remorin gene. The nucleic acid molecule of the present invention might be either introduced or introgressed into the genome of the plant. In one embodiment, the nucleic acid molecule of the present invention is foreign to the plant, i.e. it is a heterologous nucleic acid molecule.

Thus, the plant of the present invention may comprise the nucleic acid molecule according to the invention as a transgene or as endogene or may comprise the vector or expression cassette of the present invention which comprises said nucleic acid molecule as transgene. Furthermore, the plant of the present invention may comprise the cells of the present invention harboring the vector and the nucleic acid molecule of the present invention, respectively. In a preferred embodiment, the plant of the present invention is heterozygous or preferably homozygous for the nucleic acid molecule of the present invention.

In one embodiment, the plant of the present invention further comprises the Rz2 resistance gene mediating BNYVV resistance endogenously or transgenically. Accordingly, in the framework of the present invention there are provided for the first time a sugar beet plant, a chard plant, a red beet or beetroot plant, a fodder beet plant having the resistance against BNYVV mediated by the Rz2 gene and the functional remorin gene according to the invention.

A portion of the plant of the present invention may thereby be a cell, a tissue, an organ, or a combination of multiple cells, tissues, or organs. A combination of multiple organs is, for example, a blossom or a seed. A plant of the present invention comprising the Rz2 gene and the functional remorin gene according to the invention preferably shows a higher resistance against BNYVV than a corresponding plant that does contain the Rz2 gene but does not contain the nucleic acid molecule according to the invention, i.e. the functional remorin gene and/or which does contain the nucleic acid molecule of the present invention which interferes with the Rz2 mediated BNYVV resistance, i.e. the non-functional variant of the remorin (control plants). The control plants ideally have the identical genotype as the transgenic plant, and have been cultured under identical conditions, but do not contain the nucleic acid molecule of the present invention involved in Rz2 mediated BNYVV resistance and/or do contain the nucleic acid molecule of the present invention which interferes with the Rz2 mediated BNYVV resistance.

A plant cell or plant or portion thereof of the present invention that contains the nucleic acid molecule according to the invention—in particular, a plant of the genus Beta—preferably shows a higher resistance to a pathogen—in particular, to BNYVV— than a corresponding plant cell or plant or portion thereof that does not contain the nucleic acid molecule according to the invention, or contains the nucleic acid molecule of the present invention which interferes with the Rz2 mediated BNYVV resistance.

In the case of a transgenic plant cell, or plant or portion thereof, this comprises the nucleic acid molecule or DNA molecule according to the invention, i.e. the functional remorin gene, as a transgene or the vector or the expression cassette of the present invention. Such a transgenic plant cell or plant or portion thereof is, for example, one that is transformed—preferably, stably—with said nucleic acid molecule, DNA molecule, or with the corresponding vector or the expression cassette. In a preferred embodiment, the nucleic acid molecule is operatively linked with one or more regulatory sequences which allow the transcription and, optionally, the expression in the plant cell. The total structure made up of said nucleic acid molecule and the regulatory sequence(s) then represents the transgene. Such regulatory sequences are, for example, a promoter or a terminator. Numerous functional promoters and terminators that are applicable in plants are known to the person skilled in the art.

The invention also includes a vacuole of the cell according to the invention, and the content substances stored therein.

Furthermore, the invention also relates to the cell extract from a cell—preferably, from a plant cell, particularly preferably, from a cell of Beta vulgaris, and, especially preferably, from a cell of one of the following crops: sugar beet, chard, or beetroot. No plant can be regenerated from the cell extract.

Likewise encompassed by the invention is a plant genome containing the nucleic acid according to the invention, i.e. the functional remorin gene. No plant can be regenerated from the plant genome. Also encompassed by the invention is the use of the cell extract for the production of sugar (sucrose) or for the production of juice—preferably, beetroot juice. Likewise encompassed by the invention is the sugar—in particular, sucrose—contained in the cells according to the invention and their vacuoles.

An additional aspect of the invention is a seed stock comprising seeds that contain the nucleic acid molecule according to the invention, i.e. the functional remorin gene. Said nucleic acid molecule may be present transgenically or endogenously. The seed stock and the seeds may be technically treated. The invention thus also comprises technically-treated seed stock and technically-treated seeds. The various embodiments of technically-treated seed stock are explained in detail in the following whereby the term seed stock also includes seeds: Technically-treated seed stock may be present in polished form. The outermost layer of the seed is thereby removed, so that the seed assumes a more rounded form. This is helpful in sowing, where an optimally uniform shape leads to a uniform distribution of the seed stock grains. Technically-treated seed stock furthermore encompasses pelleted seed stock. The seed stock is thereby embedded in a pelleting mass that protects the seed stock contained therein and leads to a larger mass, such that the pelleted seed stock shows a greater resistance capability with regard to wind drift and is thus less susceptible to being blown away by the wind, and, at the same time, a more precise positioning during sowing is enabled. In a preferred embodiment of the invention, all pelleted seed stock grains of a batch or unit designated for sale have essentially the same shape and the same mass. Deviations of 5% in diameter and mass are possible. However, the deviations preferably do not exceed 1%. As one of the main components, the pelleting mass may contain for example a mineral compound such as clay, bentonite, kaolin, humus and/or peat, for example. It is possible to add an adhesive material like polyacrylamide. Additional possible components are cited in U.S. Pat. No. 4,067,141. Moreover, the pelleting mass may contain additional chemical agents that positively influence the cultivation in practice. These may here be substances that are counted among fertilizing agents. These include compounds rich of one or more of the following elements: Nitrogen, Phosphorus and Potassium (macronutrients). Therefore, the fertilizing ingredients may contain for example Nitrate nitrogen, Ammonium nitrogen, Magnesium Nitrate, Calcium Ammonium Nitrate, Mono Ammonium Phosphate, Mono Potassium Phosphate and Potassium Nitrate. Furthermore, pelleting mass may contain fungicides, insecticides, and/or antifeedants. The fungicides may be thiram and/or hymexazol and/or other fungicides. The insecticide may be a substance from the neonicotinoid group. The substance from the neonicotinoid group is preferably imidacloprid (ATC Code: QP53AX17) and/or clothianidin (CAS number 210880-92-5). Furthermore, the insecticide may also be cyfluthrin (CAS number 68359-37-5), beta-cyfluthrin or tefluthrin. It is worth mentioned that the compound included in the dressing or pelleting mass are taken up by the plant and show systemic effect thereby providing suitable protection of the whole plant. Plants resulting from pelleted seed including one or more pesticides therefore differ from naturally occurring plants and show better performance under biotic stress conditions. In this context the invention also encompasses a mixture of a pelleting mass and a seed according to the invention. Furthermore, the invention also encompasses a method for producing a pelleted seed according to the invention comprising the following steps:
a) providing a sugar beet plant seed comprising the nucleic acid according to the invention, i.e. the functional remorin gene,
b) embedding the sugar beet plant seed in a pelleting mass, and
c) allowing the pelleting mass to dry, wherein the seed may be optionally a primed or pregerminated seed or the seed may be allowed to be primed during step b).

The pelleted seed stock is a specific embodiment of dressed seed stock. In this context technically-treated seed stock encompasses also the dressed seed stock. However, the invention is not limited to pelleted seed stock, but, rather, may be applied with any form of dressed seed stock. The invention thus also relates to dressed seed stock, which includes pelleted seed stock, but is not limited to this. Dry dressing, wet dressing, and suspension dressing are thus also encompassed. The dressing may thereby also contain at least one dye (coloring), such that the dressed seed stock may be quickly differentiated from undressed seed stock, and, furthermore, good visibility in the environment is ensured after sowing. The dressing may also contain those agrochemicals which are described in the context of the pilling mass. The invention includes thus such dressed seed stock whereby the dressing contains at least one anti-feedant such as an insecticide and/or at least one fungicide. Optionally, so called electonical dressing (dressing by application of electric energy) may be applied. However, electronic dressing is not a dressing in the strict sense of the word.

An additional form of technically-treated seed stock is encrusted seed stock. What is known as coating is also spoken of in this context as well as of seed stock treated with a coating. The difference to pelleted seed stock is that the seed grains retain their original shape, wherein this method is especially economical. The method is described in EP 0 334 258 A1, for example. An additional form of technically-treated seed stock is sprouted or primed seed stock. Sprouted seed stock is pretreated via a pre-germination, whereas primed seed stock has been pretreated via a priming (“germination”). Pre-germinated and primed seed stock have the advantage of a shorter emergence time. The point in time of the emergence after sowing is, at the same time, more strongly synchronized. This enables better agrotechnical processing during cultivation and especially during the harvest, and, additionally, increases the yield quantity. In pre-germination, the seed stock is germinated until the radicle exits the seed stock shell, and the process is subsequently stopped. In the priming, the process is stopped before the radicle exits the seed stock shell. Compared to pre-germinated seed stock, seed stock that has been subjected to a priming is insensitive to the stress of a re-drying and, after such a re-drying, has a longer storage life in comparison to pre-germinated seed stock, for which a re-drying is generally not advised. In this context, technically pre-treated seed stock also includes primed and re-dried seed stock. The process of pre-germination is explained in U.S. Pat. No. 4,905,411 A. Various embodiments of priming are explained in EP 0 686 340 A1. In addition to this, it is also possible to simultaneously pill and prime seed stock in one process. This method is described in EP 2 002 702 B1. Primed seed stock which is moreover pelleted, is encompassed by the present invention.

The technically-treated seed stock may additionally be furnished with one or more of the herbicide resistances explained above. This allows a further-improved agrotechnical cultivation, since the technically-treated seed stock may be deployed on a field that has previously been treated with weed killer, and that therefore is weed-free.

In addition to this, the invention also encompasses a mixture containing the seed stock according to the invention or the seeds according to the invention, and a dressing mass as defined above. The dressing mass is thereby preferably embodied as a pelleting mass, as defined above.

With storage of seed stock according to the invention, storage conditions are preferably to be chosen that do not negatively affect the stability or storage life of the seed stock. Fluctuations in humidity may, especially, have a disadvantageous effect here. Part of the invention is a method for the storage of the seed stock in a bag or container that is via simultaneously water-repellent and breathable. Such a bag or container may be designed as a carton or packing. Such a carton or packing may optionally possess an inner vapor barrier. If the carton or packing is designed as a duplex carton, its stability increases. A container, bag, carton or packing comprising the seed stock according to the invention, or technically-treated seed stock according to the invention, is likewise a part of the invention. It is likewise part of the invention to store seed stock according to the invention or technically-treated seed stock according to the invention in such a bag, container, packing or carton.

In one embodiment, the plant according to the invention is a hybrid plant or a double haploid plant. Hybrid plants and double haploid plants do not occur in nature and cannot be isolated from nature. In a further embodiment of the plant according to the invention, the nucleic acid molecule according to the invention which is involved in Rz2 mediated resistance to BNYVV is present in heterozygous or more preferably in homozygous form. The invention also encompasses hybrid seeds and double haploid seeds which contain said nucleic acid molecule or the corresponding polypeptide.

A further embodiment of the present invention comprises a plant—preferably, of the species Beta vulgaris—that is characterized in that the resistance to BNYVV in this plant is further increased. For example, this may be realized by means of “gene stacking,” i.e. the resistance is increased using this dose effect. For this, the plants according to the invention that contain the BNYVV resistance-conferring Rz2 gene and the nucleic acid molecule of the present invention which is involved in the Rz2 mediated resistance are over-transformed with at least the latter one or alternative with both mentioned genes in order to increase the amount of the transcription of the gene(s) in the plant. Further possibilities for conferring, restoring or increasing the resistance to BNYVV in a plant of the species Beta vulgaris comprising the Rz2 resistance gene are described below.

An additional embodiment of the present invention relates to a sugar beet plant or a portion thereof or a pelleted seed of such a plant which is harvestable before bolting because no bolting of the sugar beet plant occurs during the first 10, 11, 12, 13, 14 or 15 months after germination and the development of a beet body is finished during this period.

In one embodiment of the present invention the sugar beet plant or a portion thereof or a pelleted seed of such a plant has a genome allowing the development of a beet body having a mass summing up to at least 50%, 60%, 70%, 80% or even 90% of the total mass of the full-grown plant.

In another embodiment of the present invention the sugar beet plant or a portion thereof or a pelleted seed of such a plant has a genome allowing the development of a beet body having a minimum mass of 200 g, 250 g, 300 g, 350 g, 400 g, 450 g or 500 g and a maximum mass of 1000 g, 1100 g, 1200 g, 1300 g, 1400 g, 1500 g, 1600 g, 1700 g, 1800 g, 1900 g or even 2000 g via photosynthesis.

An additional embodiment of the present invention is directed to a sugar beet plant or a portion thereof or a pelleted seed of such a plant wherein the genome of the sugar beet plant allows development of a beet body having a sucrose concentration of at least 10%, 11%, 12%, 13%, 14%, 15%, 16%, 17%, 18%, 19% or even 20%.

The present invention further encompasses a method for conferring, restoring or increasing the resistance to BNYVV in a plant of the species Beta vulgaris comprising the Rz2 resistance gene endogenously or transgenically mediating BNYVV resistance. Possible ways for conferring, restoring or increasing said resistance are listed in the following, however without being limited to those.

Conferring, increasing and/or restoring the resistance against BNYVV may take place via integration of the nucleic acid molecule according to the invention, i.e. the functional remorin gene, into the genome of at least one cell of a plant of the species Beta vulgaris, as well as possible regeneration of a plant from the plant cell. The integration may take place both by means of sexual crossing, e.g., crossing of a plant of the genus Beta comprising the Rz2 resistance gene mediating BNYVV resistance endogenously or transgenically with a plant comprising the functional remorin gene of the present invention, optionally in combination with the Rz2 resistance gene, and subsequent selection, and by means of homology-directed repair or homologous recombination. The two latter methods cited are preferably supported by site-directed nucleases which may be selected from, but are not limited to, the following: CRISPR nuclease, including Cas9, CasX, CasY, or Cpf1 nuclease, TALE nuclease, zinc finger nuclease, meganuclease, Argonaut nuclease, restriction endonuclease, including FokI or a variant thereof, recombinase, or two, site-specific, nicking endonucleases.

Another way for conferring, increasing and/or restoring the resistance against BNYVV may take place via transformation of a plant cell with the nucleic acid molecule according to the invention, i.e. the functional remorin gene, the corresponding recombinant DNA molecule, or with the vector or the expression cassette of the invention, and regeneration of the transgenic plant from the transformed plant cell.

In general, the activity and/or stability of the polypeptide which is encoded by the nucleic acid molecule of the present invention which modifies the Rz2 mediated resistance to BNYVV, i.e. the functional remorin gene as well as the expression of the corresponding nucleic acid molecule can be increased via modification of the nucleic acid sequence of the functional remorin gene and/or its native promoter.

The mutations leading to said modifications can be either performed within the organism itself, or it is possible to modify the corresponding nucleic acids chemically in an isolated state to achieve the desired effects. The advantage of the latter approach is that the compounds can be edited even more precisely.

The method for the production of an organism which comprises a mutated version of the nucleic acid molecule according to the above given embodiment [1] or [2] and/or a mutated version of the native promoter includes the following steps:
(I) provision of an organism or a cell comprising the nucleic acid molecule and/or the promoter;
(II) increase of the mutation rate of the organism or the cell or mutagenesis of the organism or the cell;
(III) phenotypic selection of an organism, which as a result of a mutation exhibits an altered resistance or altered resistance level towards BNYVV or genotypic selection of an organism or a cell which comprises a mutation in the nucleic acid molecule and/or the promoter, wherein the mutation has been created via step (II); and, optionally
(IV) regeneration of the organism from the cell obtained via step (III).

The organism can be a plant. Preferably the plant is a Beta vulgaris. However, it is also possible to use unicellular organisms as bacteria. The bacterium can be E. coli. If the organism is a plant then the method can be applied in vivo as well as in vitro. If the organism is a plant and the method is applied in vitro, a cell culture of the plant may be established and the increase of the mutation rate or the mutagenization may occur in the cell culture. The increase of the mutation rate encompasses for example the application of mutagenic agents like for example 5-bromouracil or ethylmethane sulfonate (EMS) or the application of physical mutagens like ionizing radiation or UV light. The mutagenization encompasses also the targeted mutagenesis. The targeted mutagenesis can be achieved by precise methods as gene editing (as explained further below). The regeneration of organism out of cells is explained in various standard references of the cell biology. The regeneration of plants is for example explained in the standard reference “Plant biotechnology: comprehensive biotechnology, second supplement” (Michael W. Fowler, Graham Warren, Murray Moo-Young—Pergamon Press—1992). The regeneration of Beta vulgaris out of the cell culture is described in Lindsey & Gallois (1990).

These references also describe how plant cell cultures are established. The mutated version of the nucleic acid molecule respectively the promoter characterizes themselves preferably due to the expression rate of the nucleic acid molecule of the present invention, i.e. the functional remorin gene which is increased by the mutation. Such an effect can also rely on the presence of several mutations. For example, it is possible to introduce two, three, four, five or more mutations in the promoter or the nucleic acid molecule.

By the introduction of mutations thus more resistance imparting protein can be built in the cell or the protein has a better effect. Thereby, the resistance in comparison to a control plant comprising the unaltered nucleic acid molecule according to the invention, i.e. the functional remorin gene, can be increased for example by at least 1, 2, 3, 4, 5 or more percent. The increase can be measured as explained further below. Moreover, the resistance due to the mutation or mutations can be increased by at least one rating score. The determination of rating scores is explained elsewhere herein. Furthermore, the protein modifying the Rz2 mediated resistance can impart—as a result of the mutations—an altered effect and in some circumstances can exhibit effect against such pathogens which have adapted themselves to the initial resistance mechanism. In this context the invention encompasses also such mutated variants of the nucleic acids according to the invention and mutated variants of the protein according to the invention. Preferably the invention encompasses such variants which do not occur in nature and cannot be isolated from nature to make sure that the pathogen had no opportunity to adapt itself to such variants. The above described method for the production of an organism which comprises a mutated version of the nucleic acid molecule may furthermore include a further step, in which those organisms or respectively plants are identified, which have a further increased resistance due to the mutation or mutations. If an increase of resistance has taken place may be determined by the herein explained rating scores or the measuring of the resistance level.

As already mentioned before, besides the above described method for the production of organisms which comprise a mutated version of the nucleic acid molecule or of the promoter it is also possible to modify the according nucleic acids chemically in an isolated state to achieve the desired effects. For this purpose, the following method is offered:

Production of a chemically modified nucleic acid molecule according to the above given embodiment [1] or [2] and/or a chemically modified promoter comprises the following steps:
(I) provision of the nucleic acid molecule as stated above in isolated form;
(II) chemical modification of the nucleic acid molecule or the promoter by one of the following steps:
(IIa) mutagenization,
(IIb) gene editing,
(IIc) restriction and ligation respectively insertion or deletion.

Furthermore, chemical modifications can be generated by such approaches as stated elsewhere herein in context of allelic variants. The gene editing given under step (II) above is equal to the term “Genome-Editing”. Optionally the chemically modified nucleic acid molecule or the chemically modified promoter can be subsequently introduced into a cell or can be stably integrated. With the help of such a cell, the chemically modified nucleic acid molecule and the modified promoter can be propagated in context of the cell proliferation. They can be subsequently isolated in vast number and expression analyses may be performed. Expression analyses are especially suitable when the chemical modification concerns the promoter. It is possible to harvest the cells and to isolate the chemically modified resistance protein for chemical analyses. If the cell which comprises the chemically modified nucleic acid molecule or the modified promoter is a plant cell, a complete plant may be regenerated out of this cell. The approaches described within this passage can be performed subsequently to the above given method for the production of a modified form of the nucleic acid molecule and/or a modified promoter and the obtained variants are also a part of this invention. Moreover, a plant comprising the chemically modified nucleic acid molecule or the modified promoter are also part of the invention. Thus, the invention is also related to a plant obtained by this method. Furthermore, the invention relates also to the chemically modified nucleic acid molecules obtained by this method and to the encoded polypeptides. These compounds may be optimized versions of the original (not modified) compounds, wherein the resulting resistance level—as explained further above—may be increased by at least by 1, 2, 3, 4, 5, or more percent or may be increased by at least one rating score. In this regard the method for the production of a chemically modified nucleic acid molecule is also a method for the optimization of the nucleic acid molecule. The method for optimization may furthermore contain an additional step, in which those modified variants of the nucleic acid molecule are identified which lead in comparison to the unamended variants to an increased resistance in a plant.

In one embodiment, increasing said resistance to BNYVV can be performed via increasing the expression of the nucleic acid molecule of the present invention which modifies the Rz2 mediated resistance to BNYVV, i.e. the functional remorin gene, in the plant. This may take place via modification of the native promoter, wherein the modification preferably takes place by means of gene editing or site-directed mutagenesis which is mediated via site-directed nucleases, and, optionally, repair models. Examples of such nucleases have already been cited above. The increase in the expression of the nucleic acid molecule according to the invention may likewise take place via fusion of the nucleic acid molecule with a heterologous promoter, which exhibits a higher activity in comparison to the native promoter—in particular, after BNYVV infection. The fusion may likewise take place via site-directed nuclease and repair models, but also by means of direct insertion after double-strand break.

As has already been mentioned above, a method for increasing the BNYVV resistance, may also be performed via increasing the activity and/or stability of the polypeptide according to the invention, via modification of the nucleotide sequence of the nucleic acid molecule according to the invention and may be performed by means of the techniques known to the person skilled in the art. This approach is explained in detail further below.

All the mentioned approaches dealing either with the introduction of the nucleic acid molecule of the present invention, i.e. the functional remorin gene, into the genome of a target plant or via modification of said nucleic acid molecule to increase its expression and the stability/activity of the corresponding protein can be optionally performed in combination with inhibiting the expression of the non-functional remorin allele.

Alternatively, a method for conferring, restoring or increasing the resistance against BNYVV might be performed via mutagenesis of a plant or plant cells comprising the nucleic acid molecule of the present invention interfering with the BNYVV resistance, i.e. the non-functional remorin allele and screening and optionally selecting for plants or plant cells in which a functional allele of the polypeptide encoded by the functional remorin allele has been restored and/or the expression of the non-functional polypeptide has been abolished. This can be performed by means of random or directed mutagenesis of the nucleic acid sequence of the non-functional remorin gene. Examples of polymorphisms which differentiate the sensitive allele from the resistant allele are presented in Tables 1 and 2.

For example, the non-functional allele may be modified via gene mutation by means of TALE nucleases (TALEN's) or zinc finger nucleases (ZFN's), as well as CRISPR/Cas systems, which—among other things—are described by way of example in WO 2014/144155 A1 (Engineering plant genomes using CRISPR/Cas systems) and in Osakabe & Osakabe, Plant Cell Physiol., 56 (2015), 389-400. This may also be achieved via use of the method designated as TILLING (Targeted Induced Local Lesions in Genomes), wherein it is described, e.g., in the German patent application DE 10 2013 101 617, how point mutations are caused in a sensitive gene, and plants are subsequently selected that exhibit a suitable, i.e., resistance-conferring, mutation, e.g., a barley resistant to yellow mosaic virus; see DE 10 2013 101 617 on pp. 4, 8, and 12, in paragraphs [0014], [0026], and [0038]. The TILLING method is also described in detail in the publication by Henikoff et al. (Henikoff et al., Plant Physiol. 135, 2004, 630-636).

In order to enable a break at a predetermined target site, the enzymes for targeted mutagenesis preferably include a binding/recognition domain and a cleavage domain. Particular enzymes capable of inducing double or single-stranded breaks are nucleases or nickases (DSBI—double strand break inducing- or SSBI—single strand break inducing—enzymes) as well as variants thereof, including such molecules no longer comprising a nuclease or nickase function but rather operating as recognition molecules in combination with another enzyme. In recent years, many suitable nucleases, especially tailored endonucleases have been developed comprising meganucleases, zinc finger nucleases, TALE nucleases, Argonaute nucleases, derived, for example, from Natronobacterium gregoryi, and CRISPR nucleases, comprising, for example, Cas9, Cpf1, Csm1, CasX or CasY nucleases as part of the Clustered Regularly Interspaced Short Palindromic Repeats (CRISPR) system. Thus, in a preferred aspect of the invention, the genome engineering component comprises a DSB- or SSB-inducing enzyme or a variant thereof selected from a CRISPR/Cas endonuclease, preferably a CRISPR/Cas9 endonuclease a CRISPR/Cpf1 endonuclease, or a CRISPR/Csm1 endonuclease, a zinc finger nuclease (ZFN), a homing endonuclease, a meganuclease and a TAL effector nuclease.

Rare-cleaving endonucleases are DSBI/SSBI enzymes that have a recognition site of preferably about 14 to 70 consecutive nucleotides, and therefore have a very low frequency of cleaving, even in larger genomes such as most plant genomes. Homing endonucleases, also called meganucleases, constitute a family of such rare-cleaving endonucleases. They may be encoded by introns, independent genes or intervening sequences, and present striking structural and functional properties that distinguish them from the more classical restriction enzymes, usually from bacterial restriction-modification Type II systems. Their recognition sites have a general asymmetry which contrast to the characteristic dyad symmetry of most restriction enzyme recognition sites. Several homing endonucleases encoded by introns or inteins have been shown to promote the homing of their respective genetic elements into allelic intronless or inteinless sites. By making a site-specific double strand break in the intronless or inteinless alleles, these nucleases create recombinogenic ends, which engage in a gene conversion process that duplicates the coding sequence and leads to the insertion of an intron or an intervening sequence at the DNA level. A list of other rare cleaving meganucleases and their respective recognition sites is provided in Table I of WO 03/004659 (pages 17 to 20) (incorporated herein by reference).

Furthermore, methods are available to design custom-tailored rare-cleaving endonucleases that recognize basically any target nucleotide sequence of choice. Briefly, chimeric restriction enzymes can be prepared using hybrids between a zinc-finger domain designed to recognize a specific nucleotide sequence and the non-specific DNA-cleavage domain from a natural restriction enzyme, such as FokI. Such methods have been described e.g. in WO 03/080809, WO 94/18313 or WO 95/09233 and in Isalan et al. (2001). A rapid, generally applicable method to engineer zinc fingers illustrated by targeting the HIV-1 promoter. Nature biotechnology, 19(7): 656; Liu et al. (1997). Design of polydactyl zinc-finger proteins for unique addressing within complex genomes. Proceedings of the National Academy of Sciences, 94(11): 5525-5530.

Another example of custom-designed endonucleases includes the TALE nucleases (TALENs), which are based on transcription activator-like effectors (TALEs) from the bacterial genus Xanthomonas fused to the catalytic domain of a nuclease (e.g. FokI or a variant thereof). The DNA binding specificity of these TALEs is defined by repeat-variable di-residues (RVDs) of tandem-arranged 34/35-amino acid repeat units, such that one RVD specifically recognizes one nucleotide in the target DNA. The repeat units can be assembled to recognize basically any target sequences and fused to a catalytic domain of a nuclease create sequence specific endonucleases (see e.g. Boch et al. (2009). Breaking the code of DNA binding specificity of TAL-type III effectors. Science, 326(5959), 1509-1512; Moscou & Bogdanove (2009). A simple cipher governs DNA recognition by TAL effectors. Science, 326(5959), 1501-1501; and WO 2010/079430, WO 2011/072246, WO 2011/154393, WO 2011/146121, WO 2012/001527, WO 2012/093833, WO 2012/104729, WO 2012/138927, WO 2012/138939). WO 2012/138927 further describes monomeric (compact) TALENs and TALEs with various catalytic domains and combinations thereof.

Recently, a new type of customizable endonuclease system has been described; the so-called CRISPR/Cas system. A CRISPR system in its natural environment describes a molecular complex comprising at least one small and individual non-coding RNA in combination with a Cas nuclease or another CRISPR nuclease like a Cpf1 nuclease or a Csm1 nuclease (Zetsche et al., “Cpf1 Is a Single RNA-Guides Endonuclease of a Class 2 CRISPR-Cas System”, Cell, 163, pp. 1-13, October 2015; US 2017/0233756 A1) which can produce a specific DNA double-stranded break. Presently, CRISPR systems are categorized into 2 classes comprising five types of CRISPR systems, the type II system, for instance, using Cas9 as effector and the type V system using Cpf1 as effector molecule (Makarova et al., Nature Rev. Microbiol., 2015). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems which have been characterized so far. Firstly, the invading DNA, also known as a spacer, is integrated between two adjacent repeat regions at the proximal end of the CRISPR locus. Type II CRISPR systems code for a Cas9 nuclease as the key enzyme for the interference step, which system contains both a crRNA and also a trans-activating RNA (tracrRNA) as the guide motif. These hybridize and form double-stranded (ds) RNA regions which are recognized by RNAseIII and can be cleaved in order to form mature crRNAs. These then in turn associate with the Cas molecule in order to direct the nuclease specifically to the target nucleic acid region. Recombinant gRNA molecules can comprise both the variable DNA recognition region and also the Cas interaction region and thus can be specifically designed, independently of the specific target nucleic acid and the desired Cas nuclease.

As a further safety mechanism, PAMs (protospacer adjacent motifs) must be present in the target nucleic acid region; these are DNA sequences which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337: 816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973 A1). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

The cleavage site of a DSBI/SSBI enzyme relates to the exact location on the DNA or RNA where the break is induced. The cleavage site may or may not be comprised in (overlap with) the recognition site of the DSBI/SSBI enzyme and hence it is said that the cleavage site of a DSBI/SSBI enzyme is located at or near its recognition site. The recognition site of a DSBI/SSBI enzyme, also sometimes referred to as binding site, is the nucleotide sequence that is (specifically) recognized by the DSBI/SSBI enzyme and determines its binding specificity. For example, a TALEN or ZNF monomer has a recognition site that is determined by their RVD repeats or ZF repeats respectively, whereas its cleavage site is determined by its nuclease domain (e.g. FokI) and is usually located outside the recognition site. In case of dimeric TALENs or ZFNs, the cleavage site is located between the two recognition/binding sites of the respective monomers, this intervening DNA or RNA region where cleavage occurs being referred to as the spacer region.

A person skilled in the art would be able to either choose a DSBI/SSBI enzyme recognizing a certain recognition site and inducing a DSB or SSB at a cleavage site at or in the vicinity of the preselected/predetermined site or engineer such a DSBI/SSBI enzyme. Alternatively, a DSBI/SSBI enzyme recognition site may be introduced into the target genome using any conventional transformation method or by crossing with an organism having a DSBI/SSBI enzyme recognition site in its genome, and any desired nucleic acid may afterwards be introduced at or in the vicinity of the cleavage site of that DSBI/SSBI enzyme.

As used herein, a “double-stranded DNA break inducing enzyme”, “enzyme inducing a double-stranded break”, or “DSBI enzyme” is an enzyme capable of inducing a double-stranded DNA break at a particular nucleotide sequence, called the “recognition site” or “predetermined site” or “predetermined location”. Accordingly, a “single-stranded DNA or RNA break inducing enzyme”, “enzyme inducing a single-stranded break”, or “SSBI enzyme” is an enzyme capable of inducing a single-stranded DNA or RNA break at a particular nucleotide sequence, called the “recognition site” or “predetermined site” or “predetermined location”.

As used herein, a “repair nucleic acid molecule” or “repair matrix” is a single-stranded or double-stranded DNA molecule or RNA molecule that is used as a template for modification of the genomic DNA or the RNA at the preselected site in the vicinity of or at the cleavage site. As used herein, “use as a template for modification of the genomic DNA”, means that the repair nucleic acid molecule is copied or integrated at the preselected site by homologous recombination between the flanking region(s) and the corresponding homology region(s) in the target genome flanking the preselected site, optionally in combination with non-homologous end joining (NHEJ) at one of the two end of the repair nucleic acid molecule (e.g. in case there is only one flanking region).

As used herein, “a modification of the genome”, means that the genome has changed in at least one nucleotide or by at least one epigenetic editing.

As used herein “a preselected site”, “a predetermined site” or “predefined site” indicates a particular nucleotide sequence in the genome (e.g. the nuclear genome or the chloroplast genome) at which location it is desired to insert, replace and/or delete one or more nucleotides.

In various embodiments, the at least one base editor according to the present invention is temporarily or permanently linked to at least one site-specific DSBI/SSBI enzyme complex or at least one modified site-specific DSBI/SSBI enzyme complex, or optionally to a component of said at least one site-specific DSBI/SSBI enzyme complex. The linkage can be covalent and/or non-covalent. Any base editor or site-specific DSBI/SSBI enzyme complex, or a catalytically active fragment thereof, or any component of a base editor complex or of a site-specific DSBI/SSBI enzyme complex as disclosed herein can be introduced into a cell as a nucleic acid fragment, the nucleic acid fragment representing or encoding a DNA, RNA or protein effector, or it can be introduced as DNA, RNA and/or protein, or any combination thereof.

The base editor is a protein or a fragment thereof having the capacity to mediate a targeted base modification, i.e., the conversion of a base of interest resulting in a point mutation of interest. Preferably, the at least one base editor in the context of the present invention is temporarily or permanently fused to at least one DSBI/SSBI enzyme, or optionally to a component of at least one DSBI/SSBI. The fusion can be covalent and/or non-covalent. Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalyzed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Goudelli et al., Programmable base editing of A·T to G·C in genomic DNA without DNA cleavage, Nature, 2017, 551(7681), 464, described adenine base editors (ABEs) that mediate the conversion of A·T to G·C in genomic DNA.

These methods preferably lead to an improvement in the resistance by at least one rating score—particularly preferably, to an improvement in the resistance by at least two, three, or more rating scores. After mutagenesis of the plant cells and subsequent regeneration of plants from the mutagenized plant cells, or mutagenesis of plants, the plants may then be identified that exhibit one or more mutations, as depicted in Tables 1 and 2, in an endogenous nucleic acid molecule. In this context, the already mentioned plant according to the invention may be characterized by that the resistance is increased by at least one rating score, preferably by at least two or more rating scores. Alternatively, the resistance of the plants according to the invention may be increased for example by at least 1, 2, 3, 4, 5 or more percent in comparison to a control plant, which does not comprise the nucleic acid according to the invention and/or which comprises the non-functional variant thereof. The increase can be measured by inoculation of one healthy leaf with an isolate of the pathogen and the determination of the infested surface after 15 days. A reduce of 5% of the infested surface corresponds to an increase of the resistance of 5%. Further parameters for the conduction of the measuring can be derived from the below given embodiment “resistance test”.

The method for production of a BNYVV-resistant plant comprising the nucleic acid molecule of the present invention, i.e. the functional remorin gene, alternatively includes, as described above, the provision of a plant of the genus Beta or a plant cell of the genus Beta comprising the Rz2 resistance gene mediating BNYVV resistance endogenously or transgenically, transformation of said plant cell with the nucleic acid molecule of the present invention, i.e. the functional remorin gene, or the corresponding vector or expression cassette and regeneration of a transgenic plant from the transformed plant cell.

In an alternative embodiment, the method for production of a BNYVV-resistant plant comprises the provision of a plant of the genus Beta or a plant cell of the genus Beta comprising the Rz2 resistance gene mediating BNYVV resistance endogenously or transgenically, the introduction of a site-directed nuclease or nickase and optionally a repair matrix or a site-directed base editor into a cell of the plant, wherein the site-directed nuclease is able to generate at least one single-strand break of the DNA or at least one double-strand break of the DNA in the genome of the cell—preferably in the nucleic acid molecule of the present invention interfering with the BNYVV resistance, i.e. the non-functional remorin, or upstream or downstream from said nucleic acid molecule—and the repair matrix comprises the nucleic acid molecule according to the invention which is involved in BNYVV resistance, i.e. the functional remorin gene, or wherein the site-directed base editor is able to generate at least one single-strand break of the DNA at one of the mentioned locations and to convert at least one nucleobase. The nucleobases which are preferably to be converted are listed in Table 1, above. The method furthermore optionally includes the cultivation of the generated cell under conditions that allows modification of the genome at the mentioned location, wherein the modification can be a replacement, a deletion and/or an insertion of at least one nucleotide. The modification might optionally occur by homology-directed repair or homologous recombination, wherein the nucleic acid molecule is integrated into the genome of the plant. However, substitution of single nucleotide might also occur with base editors, e.g. CRISPR tools; thus the repair matrix is not necessarily required. Furthermore, the regeneration of a plant from the modified plant cell is encompassed.

The method for production of a BNYVV-resistant plant comprising the nucleic acid molecule of the present invention, i.e. the functional remorin gene, alternatively includes the crossing of a plant of the genus Beta comprising the Rz2 resistance gene mediating BNYVV resistance endogenously or transgenically with a plant comprising the functional remorin gene of the present invention, optionally in combination with the Rz2 resistance gene, and identifying and optionally selecting a plant comprising the Rz2 resistance gene and the nucleic acid molecule of the present invention, i.e. the functional remorin gene both endogenously or transgenically, wherein the Rz2 resistance gene and/or said nucleic acid molecule of the present invention is present homozygously in the genome of the plant.

The present invention likewise relates to a method for the identification, and possibly the provision, of a plant of the species Beta vulgaris, of a portion or a seed thereof comprising the nucleic acid molecule of the present invention, i.e. the functional remorin gene, and which is capable of exhibiting resistance against BNYVV mediated by the Rz2 gene, characterized in that the method includes a step of the detection of the presence and/or of the expression of said nucleic acid molecule or of the encoded polypeptide in the plant or a sample, a portion or a seed thereof. Accordingly, the presence and expression, respectively, of said nucleic acid molecule indicates that the plant is capable of exhibiting Rz2 mediated resistance against BNYVV. Alternatively, the absence and lack of expression, respectively, of said nucleic acid molecule and/or the presence and/or expression of the nucleic acid molecule of the present invention interfering with the resistance against BNYVV, i.e. the non-functional remorin gene indicates that the plant is not capable of or impaired in exhibiting Rz2 mediated resistance against BNYVV. In addition, the method may include the identification and optionally selection of the plant of the species Beta vulgaris, the portion or the seed thereof comprising the nucleic acid molecule of the present invention, i.e. the functional remorin gene, which is capable of exhibiting resistance against BNYVV mediated by Rz2, if present in the genome of the plant.

In the same way, the present invention relates to a method for the identification and optionally selection of a plant of the species Beta vulgaris comprising the nucleic acid molecule of the present invention which interferes with the resistance against BNYVV, i.e. the non-functional remorin gene, characterized in that the method includes a step of the detection of the presence and/or of the expression of said nucleic acid molecule or of the encoded polypeptide in the plant or a sample, a portion or a seed thereof.

The presence and/or the expression of a nucleic acid molecule according to the invention, or of the polypeptide according to the invention, may be tested by means of standard methods known to the person skilled in the art, e.g., by means of PCR, RT-PCR, or Western Blot.

With the fine mapping, the position of the remorin gene in the genome of Beta vulgaris subsp. vulgaris has been determined, and the gene itself and the surrounding sequence regions have been identified. In particular, remorin is positioned about 0.75 cM distal to the Rz2 gene on the long arm of chromosome 3. This in turn represents the basis for the development of DNA hybridization probes or genetic markers in the target region allowing to distinguish between plants comprising the functional remorin allele and plants comprising the non-functional remorin allele and allowing to develop markers in one or more coseggregating regions which can be used to identify recombinants between the genes and thus to separate the repressor allele from the Rz2 resistance allele. The genetic position of both genes in relation to each other are shown in FIG. 7.

DNA hybridization probes may be derived from the sequence of the remorin gene and be used for the screening of genomic and/or cDNA banks of the desired organism. The probes may be used to amplify identified homologous genes via the known process of polymerase chain reaction (PCR), and to check whether the remorin gene is present endogenously in an organism, or has been successfully introduced heterologously.

The person skilled in the art may here resort to customary hybridization, cloning, and sequencing methods, which, for example, are listed in Sambrook et al., Molecular Cloning: A Laboratory Manual, 3rd ed., Cold Spring Harbor Laboratory Press, Cold Spring Harbor, N.Y., 2001. The person skilled in the art may also synthesize and use oligonucleotide primers to amplify sequences of the remorin gene. In order to achieve a specific hybridization, such probes should be specific and have at least a length of 15 nucleotides—preferably, at least 20 nucleotides. A detailed guide to hybridization of nucleic acids may be found in Tijssen, Laboratory Techniques in Biochemistry and Molecular Biology—Hybridization with Nucleic Acid Probes, Part 1, Chapter 2, “Overview of principles of hybridization and the strategy of nucleic acid probe assays.” Elsevier, New York (1993); and in Current Protocols in Molecular Biology, Chapter 2, Ausubel et al., eds., Greene Publishing and Wiley Interscience, New York (1995).

Thus, in one embodiment, the present invention relates to oligonucleotides of at least 15, 16, 17, 18, 19, or 20—preferably, at least 21, 22, 23, 24, or 25, particularly preferably, at least 30, 35, 40, 45, or 50, and, especially preferably, at least 100, 200, 300, 500 or 1,000—nucleotides in length that specifically hybridize with a nucleotide sequence of the nucleic acid molecule according to the invention which is involved in Rz2 mediated resistance against BNYVV, or that specifically hybridize with a nucleotide sequence of the nucleic acid molecule according to the invention which interferes with the resistance against BNYVV, or the nucleic acid molecules that are complementary to both nucleic acid sequences. Furthermore, the invention relates to a pair of nucleic acid molecules—preferably, in the form of oligonucleotides—that is suitable for attachment as a forward and reverse primer to a region that is specific to one of said nucleic acid molecules, and for amplifying these in a polymerase chain reaction (PCR).

The present invention thus also relates to markers as oligonucleotides—in particular, primer oligonucleotides. These comprise a nucleic acid molecule of at least 15 nucleotides in length that specifically hybridizes with a nucleotide sequence defined as in the preceding.

In particular, the present invention encompasses a pair of nucleic acid molecules—preferably, in the form of oligonucleotides or a kit containing this pair of oligonucleotides—that is suitable for hybridization as a forward and reverse primer to a region that is specific to the nucleic acid molecule according to the invention, and for amplifying this in a polymerase chain reaction (PCR), or that is suitable as a forward and reverse primer for hybridization to a region in the Beta vulgaris genome that, in Beta vulgaris, exhibits a cosegregation with the nucleic acid molecule according to the invention which is involved in Rz2 mediated resistance against BNYVV, i.e. with the functional remorin gene, or with the nucleic acid molecule according to the invention which interferes with the resistance against BNYVV, i.e. the non-functional remorin gene. Alternatively or additionally, the oligonucleotide or the pair of oligonucleotides is suitable for hybridization to a region in the Beta vulgaris genome that, in Beta vulgaris, cosegregates with the functional remorin gene together with the Rz2 gene, or with the non-functional remorin gene together with the Rz2 gene.

TABLE 3
Markers flanking remorin and the Rz2 gene as shown in FIG. 7.

| | | | nucleobases in | |
|---|---|---|---|---|
| | | | the genome of | |
| | | | Beta vulgaris | |
| | genetic | physical | comprising the | |
| marker | position | position | non-functional | SEQ ID |
| name | [cM] | [bp] | remorin gene | No: |
| s3e5985s01 | 36.9318639 | 7077769 | C | 33 |
| sxh1002s02 | 39.0210334 | 7957425 | G | 34 |
| sxh2499s01 | 39.9958561 | 8403820 | A | 35 |
| sxi0698s01 | 40.5090706 | 8648395 | T | 36 |
| sxh8485s01 | 42.3807876 | 9605012 | A | 37 |
| sxe7357s01 | 42.8377616 | 9856523 | C | 38 |
| sxn1017s02 | 43.6903085 | 10348480 | T | 39 |
| s3e2247xxx | 45.7583869 | 11699138 | A | 40 |

In a preferred embodiment, the oligonucleotide or the pair of oligonucleotides of the present invention is selected from the group consisting of those set forth in SEQ ID Nos. 24 to 40. In particular, 74 markers have been identified between remorin and Rz2, three of which are exemplified in FIG. 7: s3e5782s01 (SEQ ID NO: 30; non-functional allele: T), s3e4913xxx (SEQ ID NO: 31; non-functional allele: T) and s3e5800s01 (SEQ ID NO: 32; non-functional allele: T). FIG. 7 also shows marker positions distal to Remorin and proximal to Rz2, which define flanking areas that can be used for marker assisted breeding (MAS) with the aim of introducing a functional remorin. These markers and their respective genetic and physical positions are shown in Tables 3 to 5.

TABLE 4
Markers located between remorin and the Rz2 gene as shown
in FIG. 7.

| | | | nucleobases in | |
|---|---|---|---|---|
| | | | the genome of | |
| | | | Beta vulgaris | |
| | genetic | physical | comprising the | |
| marker | position | position | non-functional | SEQ ID |
| name | [cM] | [bp] | remorin gene | No: |
| s3e5782s01 | 41.15 | 8961406 | T | 30 |
| s3e4913xxx | 41.38 | 9079399 | T | 31 |
| s3e5800s01 | 41.73 | 9260730 | T | 32 |

TABLE 5
Markers located in the remorin gene as shown in FIG. 7.

| | | | nucleobases in | |
|---|---|---|---|---|
| | | | the genome of | |
| | | | Beta vulgaris | |
| | genetic | physical | comprising the | |
| marker | position | position | non-functional | SEQ ID |
| name | [cM] | [bp] | remorin gene | No: |
| s3p4348s01 | ~41.0 | 8886686 | G | 27 |
| s3p4349s01 | ~41.0 | 8886781 | T | 28 |
| s3p4351s01 | ~41.0 | 8891242 | T | 29 |

Furthermore, the oligonucleotide according to the invention may be connected to a fluorescent dye in order to generate a fluorescence signal, e.g., under excitation via light of the corresponding wavelength. The fluorescent dye may be fluorochrome. The oligonucleotides according to the invention may be coupled with other compounds that are suitable for generating a signal. Such oligonucleotides do not occur in nature and also cannot be isolated from nature. The following is executed to produce such marked oligonucleotides: DNA may be marked bio-orthogonally. For this, DNA may be marked in vivo or in vitro with nucleoside analogs, which, for example, may subsequently be coupled with a fluorophore per Staudinger reaction. In addition to this, DNA may also be chemically provided with fluorophores. Oligonucleotides may be marked via a phosphoramidite synthesis with fluorophores that, for example, are used in QPCR, DNA sequencing, and in situ hybridization. Furthermore, DNA may be generated enzymatically in the course of a polymerase chain reaction with fluorescent nucleotides, or be marked with a ligase or a terminal deoxynucleotidyl transferase. DNA may also be detected indirectly via a biotinylation and fluorescent avidin. For couplings, fluorescein, fluorescent lanthanides, gold nanoparticles, carbon nanotubes, or quantum dots, among other things, are used as fluorophores. One of the most commonly used fluorescent substances is FAM (carboxyfluorescein). Consequently, oligonucleotides and, in particular, primers that possess a FAM marking are encompassed by the invention. FAM is preferably present as 6-FAM, wherein—depending upon the desired wavelength of the emission and excitation—other FAM variants, e.g., 5-FAM, may, however, also be used. Examples of additional fluorescence markers are AlexaFluor, ATTO, Dabcyl, HEX, Rox, TET, Texas Red, and Yakima Yellow. Depending upon the field of use, the oligonucleotides may be furnished with modifications of the bases or of the sugar phosphate spine. Among these are, among others, amino-dT, azide-dT, 2-aminopurine, 5-Br-dC, 2′-deoxyinosine (INO), 3′-deoxy-A, C, G, 5-Met-dC, 5-OH-Met-dCN6-Met-dA, and others.

Thus, the identification method according to the invention also includes the detection of the nucleic acid molecule according to the invention, i.e. the functional remorin gene, by means of detection of at least one polymorphism between the functional remorin gene and the non-functional remorin gene, i.e. between the sequences of the nucleic acid molecule according to the invention which is involved in Rz2 mediated resistance against BNYVV and the sequences of the variant of the nucleic acid molecule according to the invention which interferes with said resistance, using molecular markers that detect one or more polymorphisms. Examples for those polymorphisms are given in Table 1. Accordingly, the presence of at least one marker locus in the nucleotide sequence of the nucleic acid molecule of the present invention, i.e. the functional remorin gene, or in a cosegregating region, indicates that the plant is capable of exhibiting Rz2 mediated resistance against BYNVV. Alternatively, the absence of a marker locus in the nucleotide sequence of said nucleic acid molecule and/or or the presence of at least one marker locus in the nucleotide sequence of the nucleic acid molecule of the present invention interfering with the resistance against BNYVV, i.e. the non-functional remorin gene or in a cosegregating region indicates that the plant is not capable of or impaired in exhibiting Rz2 mediated resistance against BNYVV. In addition, the method may include the identification and optionally selection of the plant of the species Beta vulgaris, the portion or the seed thereof comprising the nucleic acid molecule of the present invention, i.e. the functional remorin gene, which is capable of exhibiting resistance against BNYVV mediated by Rz2, if present in the genome of the plant.

Following the same principle, a plant comprising the nucleic acid molecule according to the invention which interferes with the BNYVV resistance, i.e. the non-functional remorin gene, can be identified.

In a preferred embodiment of the present invention, the non-functional remorin variant comprises the nucleic acid and amino acid sequence, respectively as defined above, i.e. having the specific nucleic acid and amino acid substitutions, respectively as shown in Tables 1 and 2 and depicted in FIGS. 2 and 3. A preferred embodiment of the method according to the invention consequently includes the detection of at least one polymorphism that is presented in FIG. 2 using molecular markers which detect the polymorphisms—in particular, diagnostic polymorphisms. This detection preferably occurs using at least one molecular marker per polymorphism—in particular, per diagnostic polymorphism. It is known to the person skilled in the art which marker techniques are to be applied to detect a corresponding polymorphism, and how molecular markers for this are constructed (see Advances in Seed Science and Technology Vol. I, Vanangamudi et al., 2008).

As already outlined above, the identification method of the invention includes a step of detecting at least one marker locus in a region cosegregating with the nucleic acid molecule of the present invention. Preferably the cosegregating region is a genomic interval in Beta vulgaris on chromosome 3 which comprises and is flanked by marker loci detectable by means of

(i) the markers s3e5985s01 (SEQ ID No. 33) and s3p4348s01 (SEQ ID No. 27), and/or

(ii) the markers s3p4351s01 (SEQ ID No. 29) and s3e2247xxx (SEQ ID No. 40), preferably by means of

(i) the markers sxh1002s02 (SEQ ID No. 34) and s3p4348s01 (SEQ ID No. 27), and/or

(ii) the markers s3p4351s01 (SEQ ID No. 29) and sxn1017s02 (SEQ ID No. 39), more preferably means of

(i) the markers sxh2499s01 (SEQ ID No. 35) and s3p4348s01 (SEQ ID No. 27), and/or

(ii) the markers sxi0698s01 (SEQ ID No. 36) and s3p4348s01 (SEQ ID No. 27), and/or

(iii) the markers s3p4351s01 (SEQ ID No. 29) and sxe7357s01 (SEQ ID No. 38), and/or

(iv) the markers s3p4351s01 (SEQ ID No. 29) and sxh8485s01 (SEQ ID No. 37), and/or

(v) the markers s3p4351s01 (SEQ ID No. 29) and s3e5853s01 (SEQ ID No. 26), and/or

(vi) the markers s3p4351s01 (SEQ ID No. 29) and s3e5800s01 (SEQ ID No. 32), and/or

(vii) the markers s3p4351s01 (SEQ ID No. 29) and s3e4913xxx (SEQ ID No. 31), and/or

(viii) the markers s3p4351s01 (SEQ ID No. 29) and s3e5782s01 (SEQ ID No. 30).

The location of the markers in visualized in FIG. 7.

The method of the present invention used for the identification of a plant of the species Beta vulgaris comprising the nucleic acid molecule of the present invention, i.e. the functional remorin gene, and which is capable of exhibiting resistance against BNYVV mediated by the Rz2 gene, if present in the genome of the plant, is characterized by optionally further comprising the detection of the presence and/or expression of the Rz2 gene or the presence of the polypeptide encoded by the Rz2 gene in the plant, the portion thereof or the seed thereof; and/or the detection of at least one marker locus in the Rz2 gene or in one or more regions cosegregating with the Rz2 gene, preferably cosegregating with a chromosomal interval comprising the nucleic acid molecule of the present invention, i.e. the functional remorin gene, and the Rz2 gene. Accordingly, the identified and optionally selected plant, the portion thereof or the seed thereof comprises said nucleic acid molecule and the Rz2 gene.

In a preferred embodiment of said method, the at least one marker locus in the nucleotide sequence of the Rz2 gene is detectable by means of the marker s3e5853s01 (SEQ ID No. 26) and wherein the in one or more regions cosegregating with the Rz2 gene are genomic intervals in Beta vulgaris on chromosome 3 which comprise and are flanked by marker loci detectable by means of

(i) the markers s3p4351s01 (SEQ ID No. 29) and s3e5853s01 (SEQ ID No. 26), and/or

(ii) the markers s3e5853s01 (SEQ ID No. 26) and s3e2247xxx (SEQ ID No. 40), preferably by means of

(i) the markers s3p4351s01 (SEQ ID No. 29) and s3e5853s01 (SEQ ID No. 26), and/or

(ii) the markers s3e5853s01 (SEQ ID No. 26) and sxn1017s02 (SEQ ID No. 39), more preferably means of

(i) the markers s3p4351s01 (SEQ ID No. 29) and s3e5853s01 (SEQ ID No. 26), and/or

(ii) the markers s3e5853s01 (SEQ ID No. 33) and sxe7357s01 (SEQ ID No. 38), and/or

(iii) the markers s3e5853s01 (SEQ ID No. 26) and sxh8485s01 (SEQ ID No. 37).

In an alternative preferred embodiment of said method, the in one or more regions cosegregating with the chromosomal interval comprising the nucleic acid molecule of the present invention, i.e. the functional remorin gene, and the Rz2 gene are genomic intervals in Beta vulgaris on chromosome 3 which comprise and are flanked by marker loci detectable by means of

(i) the markers s3e5985s01 (SEQ ID No. 33) and s3p4348s01 (SEQ ID No. 27), and/or

(ii) the markers s3e5853s01 (SEQ ID No. 26) and s3e2247xxx (SEQ ID No. 40), preferably by means of

(i) the markers sxh1002s02 (SEQ ID No. 34) and s3p4348s01 (SEQ ID No. 27), and/or

(ii) the markers s3e5853s01 (SEQ ID No. 26) and sxn1017s02 (SEQ ID No. 39), more preferably means of

(i) the markers sxh2499s01 (SEQ ID No. 35) and s3p4348s01 (SEQ ID No. 27), and/or

(ii) the markers sxi0698s01 (SEQ ID No. 36) and s3p4348s01 (SEQ ID No. 27), and/or

(iii) the markers s3e5853s01 (SEQ ID No. 26) and sxe7357s01 (SEQ ID No. 38), and/or

(iv) the markers s3e5853s01 (SEQ ID No. 26) and sxh8485s01 (SEQ ID No. 37).

The oligonucleotides or the pairs of oligonucleotides of the present invention are used for identifying and/or selecting a plant of the species Beta vulgaris capable exhibiting resistance to BNYVV mediated by Rz2, if present in the genome of the plant, or which capability of resistance to BNYVV mediated by Rz2, if present in the genome of the plant, is impaired.

Furthermore, the present invention also relates to a marker chip (“DNA chip” or microarray) which contains at least one oligonucleotide according to the invention that is suitable for detection. The marker chip is suitable for application in one or more detection methods according to the invention.

The invention likewise includes a method for production of the protein according to the invention. The method includes the provision or cultivation of a cell culture which contains the nucleic acid molecule of the present invention, i.e. the functional and non-functional remorin gene, respectively, and the subsequent expression of the corresponding protein.

Furthermore, the present invention also relates to a BNYVV-resistant plant or a portion thereof which was identified, and, if applicable, selected, via a method as described above. In particular, the present invention relates to a population of plants comprising plants that are available according to one of the methods of the invention as described above, and that preferably are resistant to rhizomania, i.e. to BNYVV infestation, and are characterized by the presence of a nucleic acid molecule according to the invention which is involved in conferring Rz2 mediated resistance against BNYVV, i.e. the functional remorin gene. The population preferably has at least 10—preferably, at least 50, more preferably, at least 100, particularly preferably, at least 500, and, particularly in agricultural farming, preferably at least 1,000—plants. The proportion of plants in the population that do not carry said nucleic acid molecule, do carry the nucleic acid molecule according to the present invention which interferes with the BNYVV resistance, i.e. the non-functional remorin, and/or are susceptible to BNYVV is preferably below 25% —preferably, below 20%, more preferably, below 15%, even more preferably, 10%, and, in particular, preferably below 5%, if present at all.

The following advantages for the breeding and development of new resistant plant lines of the genus Beta may also be achieved via the present invention. Sequence information, as well as the identified polymorphisms which allow a differentiation between functional and non-functional variants of the disclosed gene, i.e. between the alleles that are involved in Rz2 mediated BNYVV resistance and the alleles interfering with said resistance, enable the marker development directly in the gene, as described above, as well as in the upstream and downstream regions, which represents an important improvement for the plant breeder—in particular, with regard to the development of optimized elite lines without “linkage drag.” Moreover, knowledge about the sequential structure may be used for the identification of additional remorin or remorin-like genes which are homologous or orthologous, for example.

Therefore, the present invention also encompasses a method for the identification of additional nucleic acid molecules encoding polypeptides or additional proteins that are involved in Rz2 mediated resistance against BNYVV in a plant in which the polypeptide is expressed. The person skilled in the art may thereby use databases, employing suitable search profiles and computer programs for the screening for homologous sequences or for sequence comparisons. Moreover, by means of conventional molecular biology techniques, the person skilled in the art may himself derive additional DNA sequences encoding remorin proteins, and use these within the scope of the present invention. For example, suitable hybridization probes may be derived from the sequence of the nucleic acid molecule according to the invention and be used for the screening of genomic and/or cDNA banks of the desired organism. The person skilled in the art may here resort to customary hybridization, cloning, and sequencing methods, which, for example, are listed in Sambrook et al., Molecular Cloning: A Laboratory Manual, 3rd ed., Cold Spring Harbor Laboratory Press, Cold Spring Harbor, N.Y., 2001. Using known sequences, the person skilled in the art may also synthesize and use oligonucleotide primers to amplify sequences of Rz2 resistance-involved nucleic acid molecules, nucleic acid molecules encoding polypeptides or additional proteins that are involved in Rz2 mediated resistance against BNYVV in a plant, or nucleic acid molecules encoding other or new remorin alleles.

In one embodiment, the present invention therefore encompasses a method for the identification of a nucleic acid molecule which encodes a polypeptide that is involved in conferring a resistance against BNYVV in a plant of the species Beta vulgaris in which the polypeptide is expressed. The method thereby includes the comparison of the amino acid sequence of the polypeptide according to the invention with amino acid sequences from a sequence database, or with sequences of allelic variants of the polypeptide according to the invention in genotypes of the species Beta vulgaris. Furthermore, the method according to the invention includes the identification of an amino acid sequence or of an allelic variant that is at least 80% identical to the amino acid sequence of the polypeptide according to the invention, as well as the introduction of a nucleic acid molecule encoding the identified amino acid sequence or allelic variant in a plant of the species Beta vulgaris comprising the Rz2 resistance gene; expression of the nucleic acid molecule in said plant; and, optionally, subsequent verification of the resistance against BNYVV.

As described in the preceding, additional proteins being involved in Rz2 mediated BNYVV resistance or their coding genes, i.e., homologs, analogs, and orthologs, that are at least 70% —preferably, at least 80%, particularly preferably, at least 90%, especially preferably, at least 95%, or even 98%—identical to the amino acid sequence of the polypeptide which is encoded by the nucleic acid molecule according to the invention may be identified via classical bioinformatic approaches (database searches and computer programs for screening for homologous sequences).

The term, homolog(s), thereby means that the genes concerned (from two different plant species) have essentially the same function and a common ancestor, and therefore typically show a significant identity in their nucleic acid or coded amino acid sequences. However, there are also many genes that are homologous to one another, without protein sequences resulting in a meaningful paired alignment. In contrast to this, the term, analog(s), describes genes or proteins that (likewise) have an identical or similar function, but are not created from the same structure, i.e., have no common ancestor. In this case, often, no significant identity can be established in their nucleic acid or encoded amino acid sequence, or, in the best case, in specific functional domains.

In the context of genome sequencing, homologs are, for annotation, more finely classified. The terms, orthology and paralogy, have been introduced for this. Orthologs are genes that are connected via a speciation event. Paralogs are genes that trace back to a duplication event.

A gene is fundamentally a homolog or analog or ortholog in the sense of the present invention if it is involved in conferring Rz2 mediated BNYVV resistance in a plant. To verify this, methods which have already been described in the preceding and known to the person skilled in the art are used, e.g., the amplification of the identified homolog or analog or ortholog by means of PCR, cloning in expression vectors, introduction into the target plant or plant cell, and checking the resistance.

The present invention also relates to the use of the nucleic acid molecule of the present invention, i.e. the functional remorin allele, in a genetic or molecular stack with other genetic elements which may confer agronomically advantageous properties to the plant. The economic value of cultivated plants may thereby be markedly increased, in that, for example, the yield performance is increased in comparison to plants that possess the same genetics, but have not been furnished with said nucleic acid molecule or which comprise the non-functional variant of the nucleic acid molecule of the present invention. In particular, the present invention relates to the use of the identified remorin gene in methods for controlling an infestation with the pathogen BNYVV in the agricultural or horticultural cultivation of plants of the genus Beta, e.g., encompassing the identification and selection of plants of the genus Beta with the aid of one of the methods described in the preceding and/or the cultivation of the plants so selected or descendants thereof. The present invention thus includes a method for the cultivation of plants of the species Beta vulgaris, including, in a first step, the provision of BNYVV-resistant plants of the species Beta vulgaris according to the invention, or the production of plants of the species Beta vulgaris with the aid of the production method according to the invention, or the identification and selection of plants of the species Beta vulgaris with the aid of the identification method according to the invention that has been described in the preceding, wherein the provision may include planting the corresponding seedlings or sowing the corresponding seeds of the present invention; and including, in a second step, the cultivation of the plants from the first step, or the deployment of seed stock of the plants from the first step, or the raising of plants from the first step. The cultivation method thereby counteracts an infestation of the cultivated plants by BNYVV. The cultivation method may be part of a method for producing sugar. The method for the production of sugar includes the steps of the cultivation method, and additionally, as a penultimate step, the harvesting of the cultivated plants, and, as a last step, the extraction of sugar from the aforesaid plants.

The cultivation method may also be part of a method for producing seed stock. The method for the production of seed stock includes the steps of the cultivation method, and additionally, as a penultimate step, the vernalization of the cultivated plants, and, as a last step, the extraction of seeds from the aforesaid plants.

The extracted seeds may optionally be pelleted, in order to obtain pelleted seed stock of the species Beta vulgaris. In this instance, it is a method for the production of pelleted seed stock. Moreover, the method for the production of seed stock may be designed as a method for the production of BNYVV-resistant seed stock. The method for the production of BNYVV-resistant seed stock includes the steps of the method described above for the production of seed stock, and additionally, as a last step, the verification of the nucleic acid according to the invention, i.e. the functional remorin gene according to a method described herein in at least one of the extracted seeds—preferably, in at least 0.1% or in at least 1% of the extracted seeds. The verification is particularly preferably implemented so that the seed remains germinable. This means that the extraction of the DNA required for verification from the seed does not neutralize the germinability of the seed. In such an instance, the verification of the nucleic acid according to the invention may have taken place in an especially large proportion of all extracted seeds. For example, the verification may take place in at least 2%—preferably, at least 3%, particularly preferably, at least 4%—of all extracted seeds.

The plants according to the invention, their cells, or seeds or seed stock according to the invention may possess additional, agronomically advantageous properties, or be furnished with such. One example is the tolerance or resistance to an herbicide such as glyphosate, glufosinate, or ALS inhibitors. The tolerance to glyphosate or an ALS-inhibitor herbicide is preferred. A specific embodiment of the glyphosate resistance is disclosed in U.S. Pat. No. 7,335,816 B2. Such a glyphosate resistance is, for example, available from seed stock stored at the NCIMB, Aberdeen (Scotland, UK), under the access number, NCIMB 41158 or NCIMB 41159. Such seeds may be used in order to obtain a glyphosate-tolerant sugar beet plant. The glyphosate resistance may also be introduced into other species of the genus Beta via crossing.

A specific embodiment of the ALS-inhibitor herbicide resistance is disclosed in the document, WO2012/049268 A1. For example, such an ALS-inhibitor herbicide resistance is available from a deposit of NCIMB, Aberdeen, UK, under the number NCIMB 41705. Furthermore, such an ALS-inhibitor resistance may be produced via tilling or site-directed mutagenesis, e.g., via gene editing, such as through the use of CRISPR/Cas, CRISPR/Cpf1, TALENS or zinc finger nucleases. Numerous additional herbicides and their applicability are known to the person skilled in the art from the prior art. He may resort to the prior art in order to achieve knowledge of which genetic elements are to be used in what manner in order to implement a corresponding tolerance in plants.

Further examples of agronomically advantageous properties are additional pathogen resistances, wherein pathogens may be amongst other insects, viruses, nematodes, bacteria, or fungi, cold tolerance or frost tolerance as well as water usage efficiency, nitrogen usage efficiency, and yield. Here, the person skilled in the art may also resort to the prior art to find suitable genetic elements.

In addition to relating to the plant according to the invention, the present invention also relates to seeds or descendants, or to an organ, a plant part, a tissue, or a cell thereof in the production of products that are typically produced from sustainable raw materials, such as foodstuffs and animal feed—preferably, sugar or syrup (molasses), wherein the molasses is also used for industrial applications, e.g., in alcohol production or as a growing medium for the production of biotechnological products, in the production of materials or substances for the chemical industry, e.g., refined chemicals, pharmaceuticals or precursors thereof, diagnostics, cosmetics, bioethanol, or biogas. An example of the use of sugar beet as a biogenic raw material in biogas plants is described in the application DE 10 2012 022 178 A1; see, for example, paragraph 10.

The following examples explain the invention, but without limiting the subject matter of the invention. Unless indicated otherwise, standard molecular biology methods have been used; see, for example, Sambrook et al., Molecular Cloning: A Laboratory Manual, 3rd ed., Cold Spring Harbor Laboratory Press, Cold Spring Harbor, N.Y., 2001, Fritsch et al., Cold Spring Harbor Laboratory Press: 1989; Mayer et al., Immunochemical Methods in Cell and Molecular Biology, eds., Academic Press, London, 1987, and Weir et al., Handbook of Experimental Immunology, Volumes I-IV, Blackwell, eds., 1986.

Some of the most important sequences according to the invention are explained in detail in the following:

SEQ ID No. 2—genomic sequence of the non-functional remorin gene (haplotype 1)

SEQ ID No. 3—open reading frame of the non-functional remorin gene (haplotype 1)

SEQ ID No. 4—amino acid sequence of the non-functional remorin polypeptide (haplotype 1)

SEQ ID No. 6—genomic sequence of the functional remorin gene (haplotype 2)

SEQ ID No. 7—open reading frame of the functional remorin gene (haplotype 2)

SEQ ID No. 8—amino acid sequence of the functional remorin polypeptide (haplotype 2)

SEQ ID No. 10—genomic sequence of the functional remorin gene (haplotype 3)

SEQ ID No. 11—open reading frame of the functional remorin gene (haplotype 3)

SEQ ID No. 12—amino acid sequence of the functional remorin polypeptide (haplotype 3)

SEQ ID No. 14—genomic sequence of the functional remorin gene (haplotype 4)

SEQ ID No. 15—open reading frame of the functional remorin gene (haplotype 4)

SEQ ID No. 16—amino acid sequence of the functional remorin polypeptide (haplotype 4)

SEQ ID No. 18—genomic sequence of the functional remorin gene (haplotype 5)

SEQ ID No. 19—open reading frame of the functional remorin gene (haplotype 5)

SEQ ID No. 20—amino acid sequence of the functional remorin polypeptide (haplotype 5)

SEQ ID No. 51—nucleotide sequence encoding Rz2

SEQ ID No. 52—amino acid sequence of Rz2 variant A

SEQ ID No. 53—amino acid sequence of Rz2 variant B

SEQ ID No. 54—nucleotide sequence including the functional remorin gene (haplotype 5), flanking regions and Rz2

SEQ ID No. 55—nucleotide sequence including the non-functional remorin gene (haplotype 1), flaking regions and Rz2

### EXAMPLES

### Example 1: Identification and Genetic Mapping of a Gene being Involved in Rz2 Mediated BNYVV Resistance, i.e. the Functional Remorin Gene, as Well as of the Variant Interfering with Said Resistance, i.e. the Non-Functional Remorin Gene

In order to localize the non-functional remorin gene leading after expression of the corresponding polypeptide to suppression of the Rz2 mediated BNYVV resistance in plants of the species Beta vulgaris, genetic mapping has been performed. For this purpose, two Beta vulgaris lines comprising the Rz2 resistance allele were crossed, wherein one parent line comprised the non-functional remorin gene and the second parent line was resistant to rhizomania. The gene of interest could then be genetically localized in the offspring population near the Rz2 locus. However, this area still contained too many genes to name a specific candidate.

Parallel to genetic mapping, the patterns of molecular markers in multigenic breeding material were analyzed. Five genotypes with a recombination between the Rz2 gene and the predicted locus containing the non-functional remorin gene were self-pollinated. Phenotypic resistance data were collected from 20 plants of the offspring. A variance analysis was performed and markers were determined. The marker s3e5798s01 (allele X: A, allele Y: G; primer allele X (SEQ ID NO: 24), primer allele Y (SEQ ID NO: 25)) showed the highest evidence for the positioning of the remorin gene. The genetic position of the marker is 40.99598509 cM on chromosome 3 of a genetic map. The physical position of the underlying SNP on the reference sequence (SBKv7) is 8,887,000 bp on chromosome 3.

Thus, this marker is genetically located only about 0.75 cM and physically about 381 kbp away from the RZ-2 resistance gene, detectable for example by marker s3e5853s01 (SEQ ID NO: 26) at genetic position 41.7471132 cM on chromosome 3 (physical position: 9,268,704 bp).

Analysis of the reference sequence for this region showed that the marker s3e5798s01 (SEQ ID NOs. 24 and 25) is located in an annotated gene model (g23238.t1), wherein the gene contains a remorin domain (C-terminal region).

Furthermore, genetic fine mapping has been performed to better resolve the region of interest. In particular recombinants were searched for which show the different allele combinations of Rz2 (BNYVV resistant) and remorin (presence of the non-function allele—yes (A)/no (B); FIG. 8). For this purpose, 2730 plants of a dividing population were analyzed with markers. There are sufficient genetic markers between the Rz2 gene and the identified remorin gene to reliably identify such recombinants; see FIG. 8. These recombinants were then tested again in the resistance test: In the test of progeny, genotypes at the H positions (=heterozygous for the non-functional allele) split 1:2:1 (A:H:B). The A and B offspring are tested as contrasts in a resistance test.

### Example 2: Functional Validation of the Remorin Gene

Several variants of transient assays for functional analysis are performed, preferably on leaf tissue. These are based on the transient gene expression of test constructs (for example plasmids, wherein the genes to be tested are under the expression control of a promoter active in plants) and the subsequent measurement of a hypersensitive response (cell death reaction) as a proxy for triggering resistance reactions. For the transfer of the test constructs into plant tissue, for example, a particle bombardment or a local transformation by means of Agrobacterium infiltration is used. Cell death reactions can be measured visually (cell collapse, yellowing, fluorescence under UV light) or by measuring the reporter gene activity of a co-transformed reporter gene (e.g. luciferase). A reduction in reporter gene activity correlates with increased cell death activity.

Assay 1: Comparison of the Cell Death Reaction after Expression of the TGB1 Gene in Two Genotypes Comprising the Resistance Allele of Rz2 (FIG. 4)

Both genotypes tested contain the resistance allele of Rz2. Genotype 1 contains the functional allele of the remorin, genotype 2 contains the non-functional allele of the remorin. After the transfer of the TGB1 gene of Beet necrotic yellow vein virus (BNYVV) (SEQ ID NOs: 41, 42) or homologous TGB1 genes, for example from Beet soil borne mosaic virus (SEQ ID NO: 43; Wetzel and Varrelmann (2018)) into the two genotypes, a cell death reaction can be measured in genotype 1. In genotype 2, a corresponding cell death reaction does not occur or its expression is significantly reduced compared to genotype 1.

This test can also be used to screen new genotypes for functional alleles or allele combinations of Rz2 and remorin. It is also potentially suitable for screening genotypes having other genes interfering with the Rz2 mediated BNYVV resistance.

Assay 2: Comparison of the Cell Death Reaction after Expression of the TGB1 Gene and Remorin Genes in Genotypes Comprising the Resistance Allele of Rz2 (FIG. 5)

A genotype comprising the resistance allele of Rz2 and the functional allele of remorin is used for the assay. In this genotype, TGB1 is transiently expressed in combination with the non-functional allele of remorin. For comparison, transient expression of TGB1 in combination with an empty vector control or transient expression of TGB1 in combination with the functional allele of remorin is performed. In the latter two combinations, cell death triggering can be measured. In comparison, the degree of cell death triggering for the combination of TGB1 and non-functional remorin allele is reduced or absent altogether.

These results indicate that the non-functional remorin allele is dominant over the functional allele of remorin. Dominant means that even in the heterozygous state (non-functional allele on one chromosome set/functional remorin allele on the other chromosome set), it develops its full effect, i.e. the non-functional allele inhibits or prevents the development of resistance in any case. Conversely, this means that Rz2 can only develop its resistance effect if the functional remorin gene is homozygous.

Assay 3: Comparison of the Cell Death Reaction after Expression of the TGB1 Gene with Combinations of Alleles of Remorin and Rz2 (FIG. 6)

The transient expression of TGB1, Rz2 and a functional or non-functional allele of remorin can take place in a genotype without Rz2 or in alternative test systems such as Nicotiana species. When functional remorin is used, a higher cell death rate can be measured compared to the use of non-functional remorin.

The test can also be used in a modified form to compare genotypes with allelic variations of remorin or Rz2. The test construct with the respective gene to be examined is kept out of the transient expression and only the other components are expressed transiently. The expression of the cell death reaction can be used as a measure for the functionality of the variable alleles.

### Example 3: Proof of Interaction Between the Polypeptide Encoded by Rz2 and the Polypeptide Encoded by TGB1 in Nicotiana benthamiana

To proof the interaction between the polypeptide of Rz2 and the viral polypeptide of TGB1 an infiltration approaches has been chosen. Two leaves per plant have been infiltrated.

### Material

### 1) Constructs

The Following Constructs have been Provided:

| Construct | Description |
|---|---|
| pZFN-nptII-70S-Rz-2_C48 | contains Rz2 resistance gene |
| FDC173 - pZFN-nptII-70S-TGB1_BNYVV | contains TGB1 gene from |
| | BNYVV |
| pZFN-nptII-70S-Rz-2_C48_D491V | positive control |
| pZFN-nptII-GG4_90-70S_4 | empty vector |

### 2) Plant Material

As plant material plants of Nicotiana benthamiana at the age of four to five weeks after germination have been provided.

### 3) Infiltration Medium

100 ml of infiltration medium have been provided by the following steps: 100 mM MgCl₂ solution has been mixed with 0.5M MES/KOH pH5.6 as well as 0.05M Acetosyringone and subsequently the end volume has been adjusted to 100 ml by adding sterile H₂O.

### Method Steps

Several cultures of Agrobacterium tumefaciens containing the above listed constructs have been provided. Each culture in LB medium of 3 ml volume. Antibiotics have been added depending on the resistance encoded by the above given constructs selected from the group consisting of: rifampicin, spectinomycin, gentamycin and kanamycin. The incubation took place over night at 28° C. on a shaker at 180 rpm.
After 18h the OD₆₀₀ value has been determined for each culture. Each liquid culture has been centrifuged for 15 min at 2500 g and room temperature. The pellet has been resuspended in LB medium without antibiotics. The volume has been adjusted to the OD value desired for infiltration: 1.
After resuspension the culture has been incubated again at 28° C. on a shaker at 180 rpm for 1 h. Afterwards the culture has been centrifuged as described above and the pellet has been resuspended in infiltration medium (composition of this medium is given above). The volume has been adjusted to the OD value desired for infiltration: 1:1 for each of the following combinations: Rz-2_C48+empty vector; Rz-2_C48+BNYVV_TGB1; empty vector+BNYVV_TGB1.
The bacterial suspension rested for 2h at room temperature. Subsequently, the different bacterial suspensions have been mixed in a relation appropriate for the infiltration: 1:1. The infiltration has been performed via an 1 ml insulin syringe on the underside of the leaf. The infiltration site as well as the infiltrated leaf have been tagged.
After infiltration the plants have been placed over night at room temperature in darkness. Afterwards, the infiltrated plants have been subjected to incubation in a climate cabinet. During each day 16h of light at 22° C. and 8h of darkness at 19° C. were applied.

### Results

The results are shown in FIG. 9. While Rz2_C48+empty vector combination and BNYVV_TGB1+empty vector combination do not lead to discoloration of the infiltrated spot, the Rz2_C48+BNYVV_TGB1 combination leads to a discoloration of the infiltrated spot due to necrosis. The necrosis show that the plant defense mechanism has been activated due the triggering of an resistance reaction.

### Example 4: Proof of Interaction Between the Polypeptide Encoded by Rz2 and the Polypeptide Encoded by TGB1 in Sugar Beet

Leaf discs of the sugar beet variety Danicia have been transiently transformed via particle bombardment with different construct combinations. Afterwards the activity of the reporter protein luciferase has been determined in the protein extract. It could be shown that the combination of Rz2_C48 with BNYVV_TGB1 triggers an immune response which comes along with cell death and a reduced activity of the luciferase reporter protein. The transformation of Rz2_C48 alone or the transformation of BNYVV-TGB1 alone did not show such a reaction which is to be interpreted that such a single component is not sufficient for triggering an immune response. However, the combination of both factors allows interaction and initiates a resistance reaction. The results are shown in FIG. 10.

### Example 5: Proof of Protein Interaction Between BNYVV_TGB1 and g23238_Remorin

Plasmids for bimolecular fluorescence complementation (BiFC) have been provided. These plasmids encoded protein fusions with split parts of the Venus fluorescence protein. We were able to observe fluorescence signals after coexpression of BNYVV-TGB1 constructs in combination with constructs comprising an allele encoding the functional variant of remorin in Nicotiana benthamiana. This fluorescence signal was not observed after expression of any of the two individual constructs. The resulting data proof the chemical interaction between the functional variant of remorin and the viral protein TGB1.

This experiment indicates a protein-protein interaction between g23238_Remorin and BNYVV_TGB1, the viral protein that activates Rz2_C48 resistance. This result is supporting data for the involvement of g23238_Remorin in the BNYVV_TGB1 detection by Rz2_C48.

1. A nucleic acid molecule encoding a polypeptide which is involved in resistance against Beet necrotic yellow vein virus (BNYVV) mediated by the Rz2 resistance gene in a plant in which the polypeptide is expressed, wherein the nucleic acid molecule comprises a nucleotide sequence which is selected from the group consisting of:
(a) a nucleotide sequence that encodes a polypeptide having the amino acid sequence according to SEQ ID No. 45;
(b) a nucleotide sequence that comprises the coding DNA sequence according to SEQ ID No. 44
(c) a nucleotide sequence that hybridizes with a complementary sequence of the nucleotide sequence according to (a) or (b) under stringent conditions;
(d) a nucleotide sequence that encodes a polypeptide which, via substitution, deletion, and/or addition of one or more amino acids of the amino acid sequence, differs from a polypeptide that is encoded by the nucleotide sequence according to (a) or (b);
(e) a nucleotide sequence that encodes a polypeptide which has an amino acid sequence that is at least 70% identical to the amino acid sequence according to SEQ ID No. 45; and
(f) a nucleotide sequence that comprises the coding DNA sequence which is at least 70% identical to the DNA sequence according to SEQ ID No. 44,
wherein the amino acid sequence of the encoded polypeptide has a proline (P) at position 25 with reference to SEQ ID No. 45, a proline (P) at position 72 with reference to SEQ ID No. 45 or a valine (V) at position 186 with reference to SEQ ID No. 45, or
wherein the nucleic acid of the coding DNA sequence has a cytosine (c) at position 73 with reference to SEQ ID No. 44, a cytosine (c) at position 214 with reference to SEQ ID No. 44, or a guanine (g) at position 556 with reference to SEQ ID No. 44, preferably
wherein the polypeptide has the amino acid sequence of SEQ ID No. 8, 12, 16, or 20, or the nucleotide sequence that encodes said polypeptide is SEQ ID No. 5, 6, 7, 9, 10, 11, 13, 14, 15, 17, 18 or 19, or the coding DNA sequence is a nucleotide sequence according to SEQ ID No. 7, 11, 15, or 19.

2. A nucleic acid molecule which specifically hybridizes to the nucleic acid molecule according to claim 1 and encodes a non-functional allele of the polypeptide, which interferes with resistance against BNYVV mediated by the Rz2 resistance gene in a plant in which the polypeptide is expressed, wherein the nucleic acid molecule preferably comprises a nucleotide sequence which is selected from
(a) a nucleotide sequence that encodes a polypeptide having an amino acid sequence according to SEQ ID No. 4 or a polypeptide having an amino acid sequence that is at least 70% identical to SEQ ID No. 4, wherein the polypeptide comprises an amino acid sequence having at least one amino acid substitution due to one or more mutations in the nucleotide sequence, preferably wherein proline (P) at position 25 with reference to SEQ ID No. 45, proline (P) at position 72 with reference to SEQ ID No. 45, and/or valine (V) at position 186 with reference to SEQ ID No. 45 is substituted with another amino acid, preferably wherein proline (P) at position 25 is substituted with alanine (A), proline (P) at position 72 is substituted with threonine (T), and/or valine (V) at position 186 is substituted with isoleucine (I); and/or
(b) a nucleotide sequence that comprises the coding DNA sequence according to SEQ ID No. 3, that comprises a coding DNA sequence which is at least 70% identical to the DNA sequence according to SEQ ID No. 3, or that hybridizes with a complementary sequence of SEQ ID No. 3 under stringent conditions, wherein the nucleotide sequence comprises at least one nucleic acid substitution due to at least one mutation leading to an amino acid substitution, preferably wherein one or more nucleotides are substituted at positions 73-75 with reference to SEQ ID No. 44, at positions 214-216 with reference to SEQ ID No. 44 and/or at positions 556-558 with reference to SEQ ID No. 44, preferably wherein cytosine (c) at position 73 is substituted with guanine (g), cytosine (c) at position 214 is substituted with adenine (a), and/or guanine (g) at position 556 is substituted with adenine (a).

3. A polypeptide which is encoded by the nucleic acid molecule according to claim 1.

4. A vector or expression cassette which comprises the nucleic acid molecule according to claim 1, preferably wherein the nucleic acid molecule is operably linked to a heterologous regulatory element.

5. A cell which comprises the nucleic acid molecule according to claim 1.

6. A plant or a portion thereof comprising the nucleic acid molecule according to claim 1 endogenously or transgenically as transgene, preferably wherein the plant additionally comprises the Rz2 resistance gene mediating BNYVV resistance endogenously or transgenically.

7. Seed or a descendant of the plant according to claim 6, wherein the seed has been technically treated, whereby the technical treatment is selected from the group consisting of:
(a) Polishing,
(b) Dressing, preferably pelleting,
(c) Incrustation, and
(d) Colouring.

8. Oligonucleotide or a pair of oligonucleotides of at least 15, 16, 17, 18, 19, or 20, preferably at least 21, 22, 23, 24, or 25, particularly preferably at least 30, 35, 40, 45, or 50, and particularly preferably at least 100, 200, 300, or 500 nucleotides in length, wherein the oligonucleotide or the pair of oligonucleotides
(i) specifically hybridizes to a nucleotide sequence as defined in claim 1; and/or
(ii) specifically hybridizes to a region in the Beta vulgaris genome that, in Beta vulgaris, cosegregates with the nucleic acid molecule according to claim 1 and/or with the nucleic acid molecule according to claim 1 together with the Rz2 gene,
preferably wherein the oligonucleotide or pair of oligonucleotides is selected from the group consisting of SEQ ID NO: 24 to 40.

9. A method for conferring, restoring or increasing the resistance to BNYVV in a plant of the species Beta vulgaris comprising the Rz2 resistance gene mediating BNYVV resistance endogenously or transgenically, including the following steps:
(i) integration or introgression of the nucleic acid molecule according to claim 1 by means of homology-directed repair or homologous recombination—preferably, promoted by site-directed nuclease—into the genome of at least one cell of a plant of the species Beta vulgaris, and optional regeneration of a plant from the plant cell; or
(ii) increasing the expression of the nucleic acid molecule according to claim 1 in the plant—preferably, via modification of the native promoter or via fusion of the nucleic acid molecule with a heterologous promoter that exhibits a higher activity in comparison to the native promoter—in particular, after BNYVV infection; or
(iii) increase in the activity and/or stability of the functional allele of the polypeptide which is encoded by the nucleic acid molecule according to claim 1 via modification of the nucleotide sequence according to claim 1;
(iv) transformation of a plant cell with the nucleic acid molecule according to claim 1, and optionally regeneration of a transgenic plant from the transformed plant cell; or
(v) and screening/selecting for plants or plant cells, respectively, in which a functional allele of the polypeptide encoded by the nucleic acid molecule according to claim 1 has been restored and/or the expression of the non-functional polypeptide has been abolished.

10. A method for producing a BNYVV-resistant plant comprising the nucleic acid molecule according to claim 1, including the following steps:
(I) provision of a plant of the genus Beta or a plant cell of the genus Beta comprising the Rz2 resistance gene mediating BNYVV resistance endogenously or transgenically; and
(IIa) transformation of the plant cell of (I) with the nucleic acid molecule according to claim 1; and
(IIb) regeneration of a transgenic plant from the transformed plant cell; or
(IIIa) introduction of a site-directed nuclease or nickase and optionally a repair matrix, or a site-directed base editor into the plant cell, wherein the site-directed nuclease is able to generate at least one single-strand break of the DNA or at least one double-strand break of the DNA at a predetermined location in the genome of the cell and the repair matrix comprises the nucleic acid molecule according to claim 1, or wherein the site-directed base editor is able to generate at least one single-strand break of the DNA at a predetermined location in the genome of the cell and to convert at least one nucleobase; and
(IIIb) optionally, cultivation of the cell from (IIIa) under conditions that allow modification of the genome at the predetermined location, selected from
a) a replacement of at least one nucleotide;
b) a deletion of at least one nucleotide;
c) an insertion of at least one nucleotide;
d) any combination of a)-c),
optionally by homology-directed repair or homologous recombination,
wherein the nucleic acid molecule is integrated into the genome of the plant; and
(IIIc) regeneration of a plant from the cell modified in (IIIb);
preferably wherein the predetermined location is in the nucleic acid molecule that specifically hybridizes to the nucleic acid molecule according to claim 1 and encodes a non-functional allele of the polypeptide or a position which is at most 10.000 base pairs upstream or downstream away from the nucleic acid molecule that specifically hybridizes to the nucleic acid molecule according to claim 1 and encodes a non-functional allele of the polypeptide; or
(IVa) crossing of the plant of (I) with a plant; and
(IVb) identifying and selecting a plant comprising the Rz2 resistance gene mediating BNYVV endogenously or transgenically and the nucleic acid molecule according to claim 1 endogenously or transgenically, wherein the Rz2 resistance gene and/or the nucleic acid molecule according to claim 1 is present homozygously in the genome of the plant.

11. A method for identifying a plant of the species Beta vulgaris, a portion thereof or a seed thereof comprising the nucleic acid molecule according to claim 1, wherein the plant is capable of exhibiting resistance against BNYVV mediated by Rz2, if present in the genome of the plant, comprising:
(i) detection of the presence and/or expression of the nucleic acid molecule according to claim 1, or the presence of the polypeptide which is encoded by the nucleic acid molecule according to claim 1 in the plant, the portion thereof or the seed thereof; and/or
(ii) detection of at least one marker locus in the nucleotide sequence of the nucleic acid molecule according to claim 1 or in one or more cosegregating regions thereof, preferably wherein the detection of the at least one marker locus in the nucleotide sequence of the nucleic acid molecule according to claim 1 comprises the use of an oligonucleotide or a pair of oligonucleotides, and wherein the one or more cosegregating regions are genomic intervals in Beta vulgaris on chromosome 3 which comprise and are flanked by marker loci detectable by means of
(a) the markers s3e5985s01 (SEQ ID No. 33) and s3p4348s01 (SEQ ID No. 27), and/or
(b) the markers s3p4351s01 (SEQ ID No. 29) and s3e2247xxx (SEQ ID No. 40); and
(iii) identification and optionally selection of the plant of the species Beta vulgaris, the portion thereof or the seed thereof comprising the nucleic acid molecule according to claim 1, which is capable of exhibiting resistance against BNYVV mediated by Rz2, if present in the genome of the plant, based on the detection of (i) and/or (ii).

12. A method for identifying a plant of the species Beta vulgaris, a portion thereof or a seed thereof comprising the nucleic acid molecule according to claim 2, comprising:
(i) detection of the presence and/or expression of the nucleic acid molecule according to claim 2, or the presence of the polypeptide which is encoded by the nucleic acid molecule according to claim 2 in the plant, the portion thereof or the seed thereof; and/or
(ii) detection of at least one marker locus in the nucleotide sequence of the nucleic acid molecule according to claim 2 or in one or more cosegregating regions thereof, preferably wherein the detection of the at least one marker locus in the nucleotide sequence of the nucleic acid molecule according to claim 2 comprises the use of an oligonucleotide or a pair of oligonucleotides, and wherein the one or more cosegregating regions are genomic intervals in Beta vulgaris on chromosome 3 which comprise and are flanked by marker loci detectable by means of
(a) the markers s3e5985s01 (SEQ ID No. 33) and s3p4348s01 (SEQ ID No. 27), and/or
(b) the markers s3p4351s01 (SEQ ID No. 29) and s3e2247xxx (SEQ ID No. 40); and
(iii) identification and optionally selection of the plant of the species Beta vulgaris, the portion thereof or the seed thereof comprising the nucleic acid molecule according to claim 2, based on the detection of (i) and/or (ii),

13. The method according to claim 11 additionally comprising
(A) detection of the presence and/or expression of the Rz2 gene or the presence of the polypeptide encoded by the Rz2 gene in the plant, the portion thereof or the seed thereof; and/or
(B) detection of at least one marker locus in the Rz2 gene or in one or more regions cosegregating with the Rz2 gene, preferably cosegregating with a chromosomal interval comprising the nucleic acid molecule according to claim 11 and the Rz2 gene;
wherein in (iii) of claim 11 the identified and optionally selected plant, the portion thereof or the seed thereof comprises the nucleic acid molecule according to claim 11, and the Rz2 gene, based on the detection of (i) and/or (ii) of claim 11 and detection of (B).

14. A method for cultivation of plants of the species Beta vulgaris, including
(i) planting seedlings of the plant according to claim 6 or sowing seeds according to claim 7, and
(ii) grown the plants from the seedlings or seeds, and
(iii) optionally, harvest root beets from the grown plants;
wherein the method counteracts an infestation of the cultivated plants with BNYVV.

15. A method of using the nucleic acid molecule according to claim 1 for the production of BNYVV-resistant plants, preferably of plants of the species Beta vulgaris, more preferably of plants of Beta vulgaris ssp. vulgaris var. vulgaris, Beta vulgaris ssp. vulgaris var. conditiva, oder Beta vulgaris ssp. vulgaris var. crassa/alba.
